(19) United States
(12) Patent Application Publication
(10) Pub. No.: US 2013/0143180 A1
(43) Pub. Date: Jun. 6, 2013

(54) PERIODONTAL TREATMENT SYSTEM AND METHOD

(71) Applicants: Douglas L. Glover, Phoenix, AZ (US); Enrico E. DiVito, Scottsdale, AZ (US); Kemmons A. Tubbs, Mesa, AZ (US); Mark P. Colonna, Whitefish, MT (US); Robert E. Barr, San Jose, CA (US)

(72) Inventors: Douglas L. Glover, Phoenix, AZ (US); Enrico E. DiVito, Scottsdale, AZ (US); Kemmons A. Tubbs, Mesa, AZ (US); Mark P. Colonna, Whitefish, MT (US); Robert E. Barr, San Jose, CA (US)

(73) Assignee: Medical Dental Advanced Technologies Group LLC, Scottsdale, AZ (US)

(21) Appl. No.: 13/633,096

(22) Filed: Oct. 1, 2012

Related U.S. Application Data
(63) Continuation of application No. 12/875,565, filed on Sep. 3, 2010, now abandoned.
Application No. 12/875,565 is a continuation-in-part of application No. 12/395,643, filed on Feb. 28, 2009, now Pat. No. 7,980,854.
Application No. 12/395,643 is a continuation-in-part of application No. 11/704,655, filed on Feb. 9, 2007, now Pat. No. 7,959,441.
Application No. 11/704,655 is a continuation-in-part of application No. 11/895,404, filed on Aug. 24, 2007, now abandoned.
(60) Provisional application No. 60/840,282, filed on Aug. 24, 2006.

(51) Int. Cl. A61N 5/06; A61B 18/22; A61C 17/00

(52) U.S. Cl. CPC A61N 5/062; A61C 17/00; A61N 5/0603; A61B 18/22
USPC 433/216

(57) ABSTRACT

Methods and apparatuses for treating a root canal in a tooth or hard and/or soft tissue within a tooth and surrounding tissues by pulsing a laser light into a reservoir, preferably after introducing liquid fluid into the reservoir, so as to disintegrate, separate, or otherwise neutralize pulp, plaque, calculus, and/or bacteria within and adjacent the fluid reservoir without elevating the temperature of any of the dentin, tooth, bones, gums, other soft tissues, other hard tissues, and any other adjacent tissue more than about 5° C.

### CROSS-REFERENCE(S) TO RELATED APPLICATION(S)

This application is a continuation-in part of pending application Ser. No. 12/395,643 entitled “Dental and Medical Treatments and Procedures,” filed on Feb. 28, 2009; pending application Ser. No. 11/704,655 entitled “Laser Based Enhanced Generation of Photoacoustic Pressure Waves in Dental and Medical Treatments and Procedures,” filed Feb. 9, 2007; and pending application Ser. No. 11/895,404 entitled “Energetically Activated Biomedical Nanotheurapeutics Integrating Dental and Medical Treatments and Procedures,” filed on Aug. 24, 2007, all of which claim priority to provisional application Ser. No. 60/840,282 entitled “Biomedically Active Nanotheurapeutics Integrating Dental and Medical Treatments and Procedures,” filed on Aug. 24, 2006. This application is also a continuation-in-part of pending U.S. provisional application Ser. No. 61/172,279 entitled “Dental and Medical Treatments and Procedures,” filed on Apr. 24, 2009. All of the above-listed applications are incorporated herein by reference in their entireties.

### FIELD

The present disclosure relates to the use of laser light and other energy sources in the field of dentistry, medicine and veterinary medicine to perform endodontic, periodontic, and other dental and medical procedures.

### BACKGROUND

Recent advances in the fields of dentistry, medicine, and veterinary medicine necessitate functional and efficient implementation of therapies during exploratory and restructuring procedures. Of specific interest is the arena of dental root canals and periodontics.

When performing root canal procedures it is desirable to efficiently debride or render harmless all tissue, bacteria, and/or viruses within the root canal system. The root canal system includes the main root canal and all of the accessory or lateral canals that branch off of the main canal. Some of these accessory canals are very small and extremely difficult to reach in order to eliminate any bacteria and/or viruses. Such accessory canals may bend, twist, change cross-section and/or become long and small as they branch off from the main canal, making them very difficult to access or target therapeutically.

An accepted dental procedure is to mechanically pull out the main canal nerve thereby separating it from the accessory canal nerves (which stay in place) then filing out the main canal with a tapered file. This action leaves an undesirable smear layer along the main canal and can plug some of the accessory canal openings, which potentially trap harmful bacteria or other harmful maladies. This is very undesirable. The dentist must chemo-mechanically debride both main and accessory canals, including the smear layer produced by the filing. Often this is done with a sodium hypochlorite solution and various other medicaments that are left in the root canal system for 30 to 45 minutes. This current methodology does not necessarily debride or render harmless all of the accessory root canals because of the difficulty in first cleaning off the smear layer then negotiating some of the smaller twisted lateral canals. As a result many treatments using this method fail over time due to reoccurring pathology. This often requires retreatment and/or sometimes loss of the tooth.

A goal of common root canal procedures is to provide a cavity which is substantially free of diseased tissue and antiseptically prepared for a permanent embalming or obturation to seal off the area. When done properly, this step enables subsequent substantially complete filling of the canal with biologically inert or restorative material (i.e., obturation) without entrapping noxious tissue in the canal that could lead to failure of the therapy.

In a typical root canal procedure, the sequence is extirpation of diseased tissue and debris from and adjacent the canal followed by obturation. Often there is an intermediate filling of the canal with a calcium hydroxide paste for sterilization and reduction of inflammation prior to obturation and final crowning. In performing the extirpation procedure, the dentist must gain access to the entire canal, shaping it as appropriate. However, root canals often are very small in diameter, and they are sometimes quite curved with irregular dimensions and configurations. It is therefore often very difficult to gain access to the full length of the canal and to properly work all surfaces of the canal wall.

Many tools have been designed to perform the difficult task of cleaning and shaping root canals. Historically, dentists have used elongate, tapered endodontic files with helical cutting edges to remove the soft and hard material from within and adjacent the root canal area. Such root canal dental procedures often result in overly aggressive drilling and filing away of otherwise healthy dentin wall or physical structure of the tooth root, thereby unduly weakening the integrity or strength of the tooth. Additionally, when performing root canal procedures, it is desirable to efficiently debride or render harmless all dead, damaged, or infected tissue and to kill all bacteria, viruses and/or other undesirable biological material within the root canal system. Illustrations of a typical root canal system are shown in FIGS. 1A and 1B. The root canal system includes the main root canal 1 and many lateral or accessory canals 3 that branch off of the main canal 1, all of which can contain diseased or dead tissue, bacteria, etc. It is common during root canal procedure to mechanically strip out the main canal nerve, often tearing it away from the lateral canal nerves, much of which can then stay in place in the canal and become the source of later trouble. Thereafter, the main canal 1 is cleaned and extirpated with a tapered file. While it is desirable to extirpate all of the main and accessory canals in a root canal system, some of the lateral canals 3 are very small and extremely difficult to reach in order to remove tissue. Such lateral canals are often perpendicular to the main canal and may bend, twist, and change cross-section as they branch off from the main canal, making them practically inaccessible to extirpation with any known file or other mechanical device. Accordingly, lateral canals are often not properly extirpated or cleaned. Many times no effort is made in this regard, relying instead on chemical destruction and embalming processes to seal off material remaining in these areas. This approach is sometimes a source of catastrophic failure that can lead to loss of the tooth and other problems. Further, when the main canal is extirpated with a tapered file, this action can leave an undesirable smear layer along the main canal which can plug some of the lateral canal openings and cause other problems that trap noxious material against later efforts to chemically disinfect the canal.

Dentists can attempt to chemo-mechanically debride and/or sterilize both main and lateral canals using a sodium hypochlorite solution or various other medicaments that are left in the root canal system for 30 to 45 minutes a time following primary mechanical extirpation of nerve and pulp tissue. However, this approach does not necessarily completely debride or render harmless all of the lateral root canals and material trapped therein because of the difficulty in cleaning off the smear layer and/or negotiating and fully wetting the solution into some of the smaller twisted lateral canals. As a result, many treatments using this method fail over time due to reoccurring pathology. This often requires retreatment and sometimes loss of the tooth.

Attempts have been made to reduce or eliminate the use of endodontic files and associated drawbacks by using lasers in the performance of root canal therapy. Some of these approaches involve burning away or carbonizing diseased and other tissue, bacteria, and the like within the canal. In these approaches, laser light is said to be directed or focused into or onto the diseased tissue, producing very high temperatures that intensely burn, carbonize, ablate, and destroy the tissue. These ablative treatments using high thermal energy to remove tissue often result in damage to the underlying collagen fibers and dentin of the root 5, even fusing the hydroxyapatite which makes up the dentin. In some cases, such treatments can cause substantial heating of the periodontal material and bone 7 surrounding the tooth, potentially causing necrosis of the bone and surrounding tissue. Additionally, the high temperatures in such treatments can melt the walls of the main canal, often sealing off lateral canals, thereby preventing subsequent treatment of lateral canals. Other attempts to use lasers for root canal therapy have focused laser light to a focal point within fluid disposed within a root canal to boil the fluid. The vaporizing fluid creates bubbles which erode material from the root canal when they implode. Such treatments which must raise the fluid temperature above the latent heat of vaporization significantly elevate the temperature of the fluid which can also melt portions of the main canal and cause thermal damage to the underlying dentin, collagen, and periodontal tissue. The damage caused to the tooth structure by these high energy ablative laser treatments weakens the integrity or strength of the tooth, similar to endodontic treatment utilizing endodontic files.

In addition to the repair of teeth through endodontic procedures, periodontal conditions such as gingivitis and periodontitis have also been treated using techniques that cause unnecessary damage to gums and tooth structure. For example, scraping techniques using dental instruments that directly remove plaque and calculus from teeth and adjacent sulcus region often remove healthy gum tissue, healthy tooth enamel, and/or cementum which is necessary for strong attachment between tooth and gum.

Therefore, there is a present and continuing need for minimally invasive, biomemetic, dental and medical therapies which remove diseased tissue and bacteria from the main root canal as well as the lateral canals of the root canal system while leaving the biological structures undamaged and substantially intact. There is also a present and continuing need for minimally invasive, biomemetic, dental and medical therapies which remove diseased tissue, plaque (including bacteria), and calculus (including bacteria) from the gums, sulcus regions, and other spaces near or between gums and teeth while leaving adjacent structures and biological cells substantially undamaged and substantially intact.

### SUMMARY

It is an object of the present invention to provide new medical, dental and veterinary devices, treatments and procedures.

In accordance with an embodiment of the present invention, a method for treating a treatment zone including one or more teeth, tissue adjacent such tooth or teeth, and a treatment pocket is provided. The method preferably comprises the steps of (A) providing a laser system containing a source of a laser light beam and an elongate optical fiber connected to said source and configured to transmit said laser light beam to a tip thereof, (B) immersing at least a portion of a tip of a light beam producing apparatus into a fluid reservoir located in the treatment pocket, the fluid reservoir holding a first fluid; and (C) pulsing the laser light source at a first setting, wherein at least a substantial portion of any contaminants located in or adjacent the treatment pocket are destroyed or otherwise disintegrated into fragmented material in admixture in and with the first fluid, thereby forming a first fluid mixture, wherein the destruction or disintegration of a substantial portion of any contaminants located in or adjacent the treatment pocket using the laser light source is accomplished without generation of any significant heat in the first fluid or associated mixture so as to avoid elevating the temperature of any gum, tooth, or other adjacent tissue more than about 5° C. In one embodiment, the first setting of step (C) further comprises an energy level of from about 2.0 W to about 4.0 W, a pulse width of from about 50 μs to about 300 μs, and a pulse frequency of from about 2 Hz to about 50 Hz. In another embodiment, the first setting of step (C) further comprises a power level of from about 10 mJ to about 100 mJ, a pulse width of from about 50 μs to about 300 μs, and a pulse frequency of from about 2 Hz to about 50 Hz. In yet another embodiment, step (B) further comprises the step of introducing the first fluid into the treatment pocket in an amount sufficient to provide a fluid reservoir and step (C) further comprises removing substantially all of the first fluid mixture from the treatment pocket. Preferably, step (C) further comprises destroying or otherwise disintegrating a substantial portion of any contaminants located in or adjacent the treatment pocket using the laser without generation of any significant heat in the first fluid so as to avoid elevating the temperature of any gum, tooth, or other adjacent tissue more than about 3° C.

In a related embodiment, step (C) further comprises the substeps of (1) removing calculus deposits in or proximate the treatment pocket by pulsing the light source at an energy level of from about 10 mJ to about 100 mJ and at a pulse width of from about 50 μs to about 300 μs, at a pulse frequency of from about 2 Hz to about 50 Hz, and moving an optical fiber used to channel the pulsed light beam in a first pattern, wherein the optical fiber includes a thickness of from about 400 microns to about 1000 microns, and wherein a substantial portion of any calculus deposits located in or proximate the treatment pocket are disintegrated into fragmented material in admixture in and with the first fluid mixture, thereby forming a second fluid mixture; and (2) optionally repeating step (C)(1) up to about six repetitions to remove substantially all calculus deposits from the treatment pocket. Step (C) may further comprise the substep of (3) modifying the surface of dentin proximate the treatment pocket by pulsing the light beam producing apparatus at a energy level of from about 0.2 W to about 4 W, a pulse width of from about 50 μs to about 300 μs, and a pulse frequency of from about 2 Hz to about 50 Hz, and moving the optical fiber in a third pattern, wherein the optical fiber includes a thickness of from about 400 microns to about 1000 microns, and wherein the tip of the laser substantially remains in contact with the tooth during pulsing and wherein the tip of the laser is maintained substantially parallel to a root of an adjacent tooth during pulsing.

In a related embodiment step (C)(3) further comprises removing remaining diseased epithelial lining to a point substantially at the base of the pocket prior to modifying the surface of the dentin by pulsing the light beam producing apparatus at the first setting wherein the first setting comprises settings selected from the group including (a) a power level of from about 10 mJ to about 100 mJ, a pulse width of from about 50 μs to about 300 μs, and a pulse frequency of from about 2 Hz to about 50 Hz; or (b) an energy level of from about 0.2 W to about 4.0 W and a continuous wave setting; wherein the optical fiber has a thickness ranging from about 400 microns to about 1000 microns. Additionally or alternatively, the method may further include the step of (C)(4) removing substantially all remaining diseased epithelial lining to a point substantially at the base of the pocket by pulsing the light beam producing apparatus at an energy level of from about 2.0 W to about 3.0 W, a pulse width of from about 50 μs to about 150 μs, and a pulse frequency of from about 2 Hz to about 50 Hz, and wherein the optical fiber includes a thickness of from about 300 microns to about 1000 microns.

In one embodiment, the method further comprises the step of (D) inducing a fibrin clot by inserting the optical fiber to about 75% the depth of the pocket, pulsing the light beam producing apparatus at an energy level of from about 3.0 W to about 4.0 W, a pulse width of from about 600 μs to about 700 μs (LP), and a pulse frequency of from about 15 Hz to about 20 Hz, and wherein the optical fiber has a diameter of from about 300 microns to about 600 microns, and, for a period of about 5 seconds to about 60 seconds, moving the optical fiber in a curved motion while slowly drawing out the optical fiber. Alternatively or additionally, the method further includes the step of (E) placing a stabilizing treatment structure substantially on one or more locations treated by the light beam producing apparatus.

In yet another embodiment, step (C)(4) occurs before step (C)(3). In this embodiment, a further step may include, for example, the additional step of (D) dissecting fibrous attachment between bone tissue and periodontal tissue along a bony defect at the base of the pocket by pulsing the light beam producing apparatus at an energy level of from about 0.2 W to about 4.0 W, a pulse width of from about 50 μs to about 600 μs, and a pulse frequency of from about 2 Hz to about 50 Hz, and wherein the optical fiber has a diameter of from about 400 microns to about 1000 microns. This embodiment, for example, may further include the step of (E) penetrating the cortical tissue of the bony defect adjacent the pocket to a depth of about 1 mm into the cortical tissue to form one or more perforations. This embodiment, for example, may further include the step of (F) inducing a fibrin clot by inserting the optical fiber to about 75% the depth of the pocket, pulsing the light beam producing apparatus at an energy level of from about 3.0 W to about 4.0 W, a pulse width of from about 600 μs to about 700 μs (LP), and a pulse frequency of from about 15 Hz to about 20 Hz, and wherein the optical fiber has a diameter of from about 300 microns to about 600 microns, and, for a period of about 5 seconds to about 60 seconds, moving the optical fiber in a curved motion while slowly drawing out the optical fiber. This embodiment, for example, may further include the step of (G) placing a stabilizing treatment structure substantially on one or more locations treated by the light beam producing apparatus.

In an alternative embodiment, step (C) further comprises the substeps of (1) removing at least a portion of the epithelial layer of a treatment zone by pulsing the light beam producing apparatus at the first setting wherein the first setting comprises settings selected from the group consisting of (a) a power level of from about 10 mJ to about 200 mJ, a pulse width of from about 50 μs to about 300 μs, and a pulse frequency of from about 2 Hz to about 50 Hz, (b) an energy level of from about 0.2 W to about 4.0 W, a pulse width of from about 50 μs to about 150 μs, and a frequency of from about 10 Hz to about 50 Hz, (c) an energy level of from about 0.4 W to about 4.0 W and a continuous wave setting, and moving an optical fiber used to channel the pulsed light beam in a first pattern, wherein the optical fiber has a diameter of from about 300 microns to about 1000 microns, and wherein a substantial portion of any diseased epithelial tissue located in or adjacent the epithelial layer are destroyed or otherwise disintegrated into fragmented material in admixture in and with the first fluid, thereby forming a second fluid mixture; (2) removing calculus deposits in or proximate the treatment pocket by pulsing the light beam producing apparatus at an energy level of from about 10 mJ to about 100 mJ and at a pulse width of from about 50 μs to about 300 μs, at a pulse frequency of from about 2 Hz to about 50 Hz, and moving the optical fiber in a second pattern, wherein the optical fiber has a diameter of from about 400 microns to about 1200 microns, and wherein a substantial portion of any calculus deposits located in or proximate the treatment pocket are disintegrated into fragmented material in admixture in and with the second fluid mixture, thereby forming a third fluid mixture; and (3) optionally repeating step (C)(2) up to about six repetitions to remove substantially all calculus deposits from the treatment pocket.

In accordance with another embodiment of the present invention, a light energy system for treating periodontal tissue is disclosed. In a preferred embodiment, the light energy system comprises a light source for emitting a light beam and an elongate optical fiber connected adjacent the light source configured to transmit the light beam to a tip of the optical fiber, the tip containing a tapered configuration extending to an apex with a surrounding substantially conical wall, substantially the entire surface of which is uncovered so that the light beam is emitted therefrom in a first pattern during activation of the light energy system light beam, wherein the optical fiber contains cladding in the form of a continuous sheath coating extending from a first location along optical fiber to a terminus edge spaced proximally from the apex of the tapered tip toward the light source by a distance of from about 0 mm to about 10 mm so that the surface of the optical fiber is uncovered over substantially the entirety of the tapered tip and over any part of an outer surface of the optical fiber between the terminus edge and a first edge of the tapered tip. In one embodiment, the light energy system comprises a light beam including a substantially omnidirectional pattern. In a related embodiment, the light energy system further comprises a laser beam.

### BRIEF DESCRIPTION OF THE DRAWINGS

Further features, aspects, and advantages of the present disclosure will become better understood by reference to the following detailed description, appended claims, and accompanying figures, wherein elements are not to scale so as to more clearly show the details, wherein like reference numbers indicate like elements throughout the several views, and wherein:

FIGS. 1a and 1b illustrate a root canal system including a main or primary root canal and lateral and sub-lateral canals that branch off of the main canal. Some of these lateral canals are very small and extremely difficult to reach in order to eliminate any bacteria and/or viruses. Such lateral canals may bend, twist, change cross-section and/or become long and small as they branch off from the main canal, making them very difficult to access or target therapeutically.

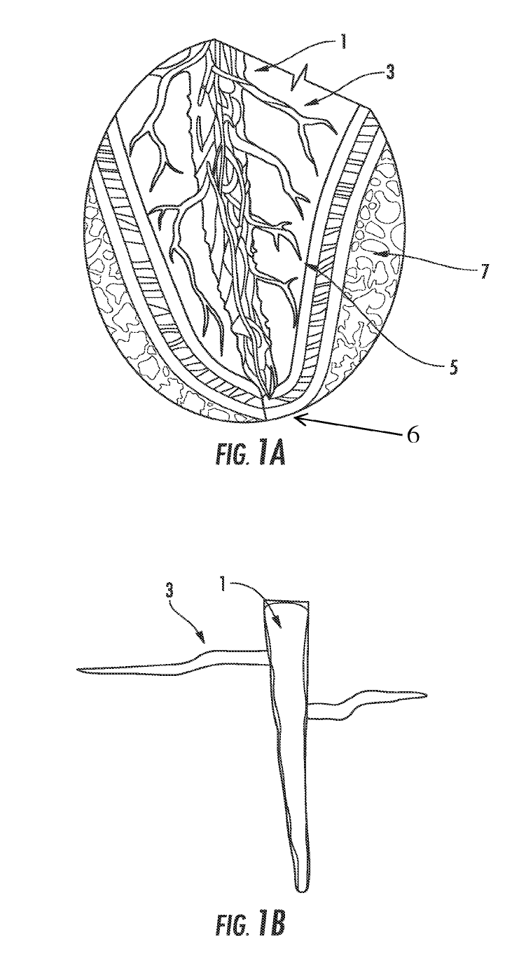

FIG. 2 is a Scanning Electron Micrograph (SEM) clearly illustrating internal reticular canal wall surfaces following use of the present invention which, as can be seen, are preserved with no burning, melting, or other alteration of the canal wall structure or loss of its porosity after subtraction of the internal tissue. The surfaces retain high porosity and surface area and are disinfected for subsequent filling and embalming, i.e. using rubber, gutta-percha, latex, resin, etc.

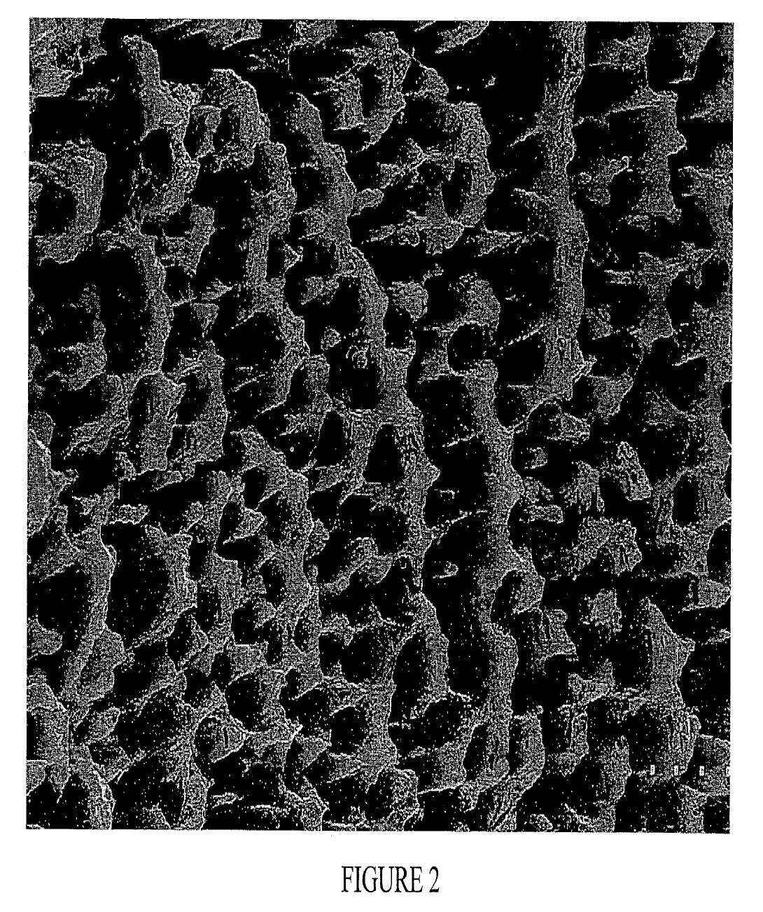

FIG. 3 is a graphical illustration of features of a laser fiber tip configured according to a preferred embodiment of the present invention.

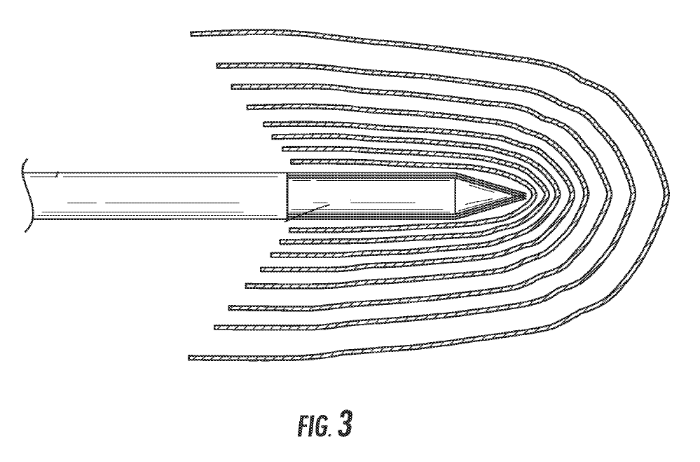

FIG. 4 is a graphical illustration of a laser system according to an embodiment of the present invention.

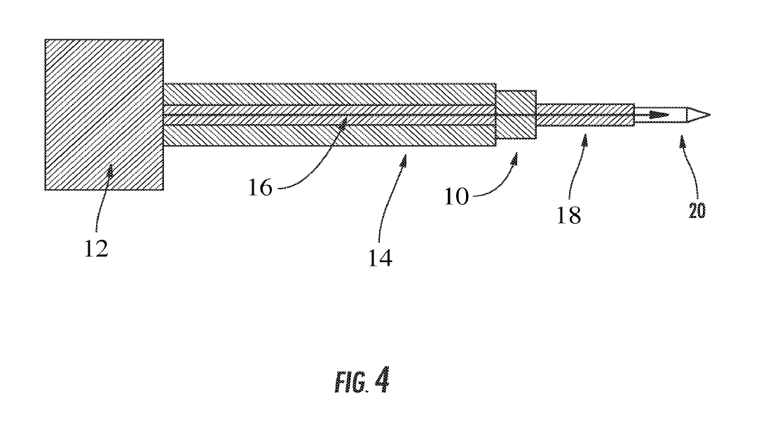

FIG. 5 is a graphical illustration of an applicator tip of a laser system according to an embodiment of the invention.

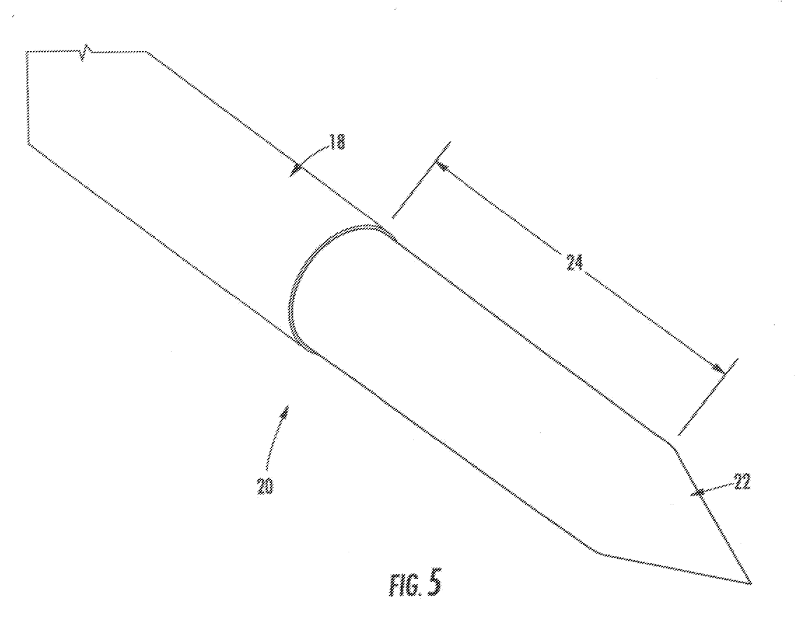

FIG. 6 shows a somewhat schematic cutaway view of a tooth and healthy surrounding gum tissue.

FIG. 7 shows a somewhat schematic cutaway view of a tooth and surrounding gum tissue including calculus deposits and partially diseased epithelium.

FIG. 8 shows a somewhat schematic cutaway view of a tooth and surrounding gum tissue including a sulcus filled with a fluid mixture in which an instrument has been inserted for treatment.

### DETAILED DESCRIPTION

Certain embodiments of the present invention are useful for treating dental, medical, and veterinary problems; primarily dental surface preparations. The present invention uses enhanced photoacoustic wave generation in dental, medical, and veterinary application during procedures that otherwise face reoccurring infection, inefficient performance and at an increase in expenses. The result of this invention has the potential to increase the effective cleaning of the root canal and accessory canals and the potential to reduce future failures over time.

A preferred embodiment utilizes an energy source which is preferably a pulsed laser energy that is coupled to a solution in such a fashion that it produces an enhanced photoacoustic pressure wave. The laser light is delivered using a commercially available laser source 10 and an optical fiber 15 attached at a proximate end to the laser source 10 and which has an application tip 20 at the distal end. The application tip 20 may be flat or blunt, but is preferably a beveled or tapered tip having a taper angle 22 between 10 and 90 degrees. Preferably any cladding 24 on the optic fiber is stripped from approximately 2-12 mm of the distal end. The taper angle of the fiber tip 20 and removal of the cladding provide wider dispersion of the laser energy over a larger tip area and consequently produces a larger photoacoustic wave. The most preferred embodiment of the application tip includes a texturing 26 or derivatization of the beveled tip, thereby increasing the efficacy of the conversion of the laser energy into photoacoustic wave energy within the solution. It should be noted that this tapered tip, the surface treatment, and the sheath stripping is not for the purpose of diffusing or refracting the laser light so that it laterally transmits radiant optical light energy to the root surface. In the current invention these features are for the sole purpose of increasing the photoacoustic wave.

Herein derivatization means a technique used in chemistry that bonds, either covalently or non-covalently, inorganic or organic chemical functional group to a substrate surface.

It was found that the photoacoustic coupling of the laser energy to the solution provides enhanced penetration of the solution into the root canal and accessory canals, thereby allowing the solution to reach areas of the canal system that are not typically accessible.

The photoacoustic (PA) wave is generated when the laser energy transitions from the tip (usually quartz or similar material) of the laser device into the fluid (such as water, EDTA, or the like). The transmission from one medium to another is not 100% efficient and some of the light energy is turned into heat near the transition that the light makes from one media to the other. This heating is very rapid, locally heating some of the molecules of the fluid very rapidly, resulting in molecule expansion and generating the photoacoustic wave. In a pulsed laser, a wave is generated each time the laser is turned on, which is once per cycle. A 10 Hz pulsed laser then generates 10 waves per second. If the power level remains constant, the lower the pulse rate, the greater the laser energy per pulse and consequently the greater the photoacoustic wave per pulse.

A method and apparatus according to a preferred embodiment of the present invention uses a subablative energy source, preferably a pulsing laser, to produce photoacoustic energy waves in solutions dispensed in a root canal of a tooth and/or sulcus adjacent such tooth to effectively clean the root canal and lateral canals and/or tissue adjacent the tooth and exterior tooth structure. In the context of this application, the term “subablative” is used to refer to a process or mechanism which does not produce or cause thermal energy-induced destruction of nerve or other native tooth structure, material or tissue, namely, that does not carbonize, burn, or thermally melt any tooth material. The pulsing laser in the inventive configuration of a preferred embodiment induces oscillating photoacoustic energy waves which emanate generally omnidirectionally from adjacent the exposed length of an applicator tip where light energy is caused to exit the surface of optical fiber material in many directions/orientations into adjacent fluid medium from a light energy source maintained at a relatively low power setting of from about 0.1 to no more than about 1.5 watts for endodontic treatment and from about 0.4 watts to about 4.0 watts for periodontal treatment in order to avoid any ablative effects.

According to one embodiment of the present invention, a tooth is first prepared for treatment in a conventional manner by drilling a coronal access opening in the crown of the tooth to access the coronal or pulp chamber and associated root canal. This may be performed with a carbide or diamond bur or other standard approaches for preparation of a tooth for root canal treatment known in endodontic practice after which the upper region above the entry of the canal into the chamber is generally emptied of pulp and other tissue. Thereafter, a first solution is slowly dispensed into the chamber, such as by use of a syringe or other appropriate mechanisms, with a small amount seeping and/or injected down into the individual root canals containing the as-yet unremoved nerves and other tissue. The first solution is preferably dispensed in an amount sufficient to fill the chamber to adjacent the top of the chamber. In other embodiments, portions of the nerve and other tissue in the canals may be removed using a broach or other known methods for removing a nerve from a root canal before the first solution is dispensed into the chamber and down into the root canals. In some embodiments, only a single solution may be used, although multiple solutions or mixtures may also be used as explained in more detail below.

The first solution preferably includes a compound containing molecules with at least one hydroxyl functional group and/or other excitable functional groups which are susceptible to excitation by a laser or other energy source in the form of rapidly oscillating photoacoustic waves of energy to assist with destructive subablative disintegration of root canal nerve tissue. It has been observed that certain fluids which do not contain excitable groups, such as xylene, do not appear to produce the desired photoacoustic wave when an energy source has been applied. In one embodiment of the invention, the first solution is a standard dental irrigant mixture, such as a solution of water and ethylenediamine tetraacetic acid (EDTA), containing hydroxyl or other excitable groups. In other embodiments of the invention, the hydroxyl-containing solution may be distilled water alone. In other alternate embodiments, solutions containing fluids other than water may be used, or various pastes, perborates, alcohols, foams, chemistry-based architectures (e.g. nanotubes, hollow spheres) and/or gels or a combination of the like may be used. Additionally, various other additives may be included in the solution. For example, and not by way of limitation, the first solution may include agents energizable by exposure to energy waves propagated through the solution from adjacent the fiber. These include materials selected from the group consisting of hydrogen peroxide, urea hydrogen peroxide, perborates, hypochlorites, or other oxidizing agents and combinations thereof. Additional additives believed to be energizable in the solution include materials selected from the group consisting of reducing agents, silanols, silanating agents, chelating agents, chelating agents coordinated or complexed with metals (such as EDTA-Calcium), anti-oxidants, sources of oxygen, sensitizing agents, catalytic agents, magnetic agents and rapidly expanding chemical, pressure or phase change agents and/or combinations of the like. The solution may also include dispersions or mixtures of particles containing nano- or micro-structures, preferably in the nature of fullerenes, such as nanotubes or bucky balls, or other nanodevices (including micro-sized devices) capable of sensitizing or co-acting with oxygenating, energizable, or activatable components in the solution/mixture, such as oxidative bleaching or other oxygenated agents. Various catalytic agents may be titanium oxide or other similar inorganic agents or metals. The first solution may also include additional effective ingredients such as surfactants or surface active agents to reduce or otherwise modify the surface tension of the solution. Such surface active agents may be used to enhance lubrication between the nerves and other intracanal tissue and the canals wall, as well as antibiotics; stabilizers; antiseptics; anti-virals; germicidals; and polar or non-polar solvents; and the like. It is especially preferred that all materials used in the system be bio-compatible and FDA and otherwise approved, as necessary, for use in dental procedures. The amounts of any of the foregoing and other additives are generally very small in the order of a few percent by weight or only small fractions of percents. The majority of the solution/mixture is preferably water, preferably sterile triple distilled water for avoidance of undesirable or unaccounted for ionic effects.

An activating energy source is applied to the first solution contained in the coronal pulp chamber. In a preferred embodiment, the activating energy source is a pulsing laser 10. The laser light energy 16 is delivered using a laser source 12 and an optical fiber 14 attached at its proximate end to a laser source 12 and having an applicator tip 20 adjacent its distal end. The optical fiber 14 preferably has a diameter of from about 200 microns to about 400 microns. The diameter should be small enough to easily fit into the coronal pulp chamber and, if necessary, into a root canal itself, but large enough to provide sufficient energy via light carried therein to create a photoacoustic effect and to prevent avoidable leakage of light or loss of energy and damage to the tooth or the fiber tip. In a preferred embodiment, the laser source is a solid state laser having a wavelength of from about 700 nm to about 3000 nm, such as NdYAG, ErYAG, HoYag, NdYLF, Ti Sapphire, or ErCrYSGG laser. However, other suitable lasers sources may be used in various embodiments.

An appropriately dimensioned laser applicator tip 20 is preferably placed into the coronal chamber until it is at least fully immersed in the first solution. By “fully immersed” it is meant liquid level is even with the edge of the cladding or other covering on the optical fiber 18. Preferably, the distal most edge of any cladding or covering 18 on the optical fiber 18 adjacent the tip is spaced approximately 2-10 mm from the distal end of the distal end tip or end of the optical fiber, most preferably about 5 mm therefrom. As a result, up to about 10 mm and most preferably about 5 mm of the distal end of the optical fiber is uncovered. In other embodiments, however, the distal most edge of any cladding or covering 18 on the optical fiber adjacent the tip is substantially at the distal end of the distal end tip or end of the optical fiber. Preferably, all or substantially all of the length of this uncovered part of the tip end is immersed. If the uncovered part of the applicator tip is not fully immersed, sufficient energy may not be transferred to the fluid since light will be permitted to escape to the environment above the liquid surface. Accordingly, it is believed that spacing the distal-most or outermost end edge of the cladding more than about 10 mm should be avoided, as that can diminish the effectiveness of the system. In some applications, it may be necessary to provide a dam and reservoir around and above the opening in the tooth in order to increase the volume and level of fluid available for immersion of the uncovered area of the end of the optical fiber. The larger liquid volume and deeper immersion of the uncovered area of the tip end is believed to enable application of sufficient energy levels to produce the desired photoacoustic wave intensity in such instances. Such instances may include, for example, smaller teeth such as upper/lower centrals or teeth that are fractured off. In certain applications where a dam or reservoir is used it may be desirable to use a laser tip with more than 10 mm of space between the tip end and the cladding due to the larger volume of fluid.

It is a feature of the invention in a preferred embodiment that the distal-most end of the applicator tip be tapered to an end point, i.e. that the distal end have a “tapered tip” 22. Most preferably, the tapered tip has an included taper angle of from about 25 to about 40 degrees. The applicator tip 20 is therefore preferably not a focusing lens configured to concentrate light to a point in space away from the tip end. Such a configuration is believed to cause an ablative effect due to the high thermal energy created by the laser light focused to a point. Rather, the taper angle of the tapered fiber tip 22 and rearward spacing of the end of the cladding from the tip end in accordance with preferred embodiments of the invention are believed to enable a relatively wide dispersion of the laser energy for emission from a relatively large surface area of the tip all the way back to the edge of the cladding, not merely from the end of the laser fiber. An objective is to emit laser light generally omnidirectionally from the sides 24 and from the tapered area 22 of the tapered applicator tip, and consequently, to produce a larger or more omnidirectional photoacoustic wave propagating into surrounding liquid and adjacent material from substantially the entire exposed surface of the fiber optic quartz material. Among other things, this avoids and preferably eliminates any ablative effects associated with higher levels of focused or refracted radiant laser energy. The tip design in accordance with the invention is selected to provide a magnitude and direction of the photoacoustic wave in the surrounding fluid medium that exhibits a relatively sharp or high rise time at the leading edge of each pulse and which propagates through the fluid generally omnidirectionally from the exposed area of the end of the fiber. Accordingly, a tapered tip according to the invention has the effect of dispersing the laser energy over the larger uncovered cone surface area and the rearwardly extending cylindrical wall surface (compared to a two dimensional generally flat circular surface area of a standard tip), thereby creating a much larger area through which the leading edges of the successive photoacoustic waves can propagate. In some embodiments, the exposed area of the fiber adjacent the tip end may include a texturing, such as frosting or etching, to increase the surface area and angular diversity of light emission for an even more comprehensive coverage of the photoacoustic wave energy within the solution and adjacent tissue.

When applying the laser to the first solution, applicants have discovered that it may be important to apply the laser energy to the solution so as to limit the creation of thermal energy. In the present invention, after the applicator tip is immersed in the first solution, laser energy is preferably applied to the first solution using subablative threshold settings, thereby avoiding any thermal-induced carbonization, melting, or other effects caused by a temperature rise above about 5° C. in the dentin walls of the canal, apical portions of the tooth, or surrounding bone or tissue caused by the generation of significant thermal energy in the canal area or wall due to the ablative power settings used in prior attempts to perform root canal therapy with lasers. The practice of the present invention in accordance with its preferred embodiments causes an observable temperature rise in the solution of no more than a few degrees Centigrade and, as a result, no more than a few degrees Centigrade elevation, if any, of the dentin wall and other adjacent tooth structure and tissue. This is far below the standard constraint of avoiding any exposure of such material and tissue to more than 5° C. increase in temperature for any significant period of time to avoid permanent damage in the same.

The inventors have found that relatively low power settings of from about 0.1 watt to about 1.5 watt and with a laser pulse duration of from about 100 nanoseconds to about 1000 microseconds, with a pulse length of about 50 microseconds most preferred, produces the desired photoacoustic effect without heating the fluid or surrounding tissue to produce any ablative or other thermal effect within or adjacent the root canal. A frequency of from about 5 to 25 Hz is preferred and a frequency of about 15 Hz is believed to provide optimal potentiation of harmonic oscillation of pressure waves in the fluid medium to disintegrate nerve and other tissue within the canal.

With regard to periodontal embodiments, the inventors have found that relatively low power settings of from about 0.4 watts (W) to about 4.0 W and with a laser pulse duration of from about 100 nanoseconds to about 1000 microseconds (μs), with a pulse length of from about 50 μs to about 650 μs most preferred, produces the desired photoacoustic effects without heating fluid located in the sulcus or surrounding tissue to produce any ablative or other thermal effect within or adjacent the sulcus. Typically, a frequency of from about 15 hertz (Hz) to about 25 Hz is preferred and a frequency of about 2 Hz to about 50 Hz is believed to provide optimal potentiation of harmonic oscillation of pressure waves in a fluid medium to destroy plaque and to disintegrate calculus in the sulcus and/or calculus attached adjacent a tooth. Preferred energy input preferably ranges from about 10 millijoules (mJ) to about 300 mJ.

The particular preferred power level found to produce the ideal photoacoustic wave has a relationship to the approximate root volume of a particular tooth. The following chart (Table 1) shows what are believe to be preferred ranges of power levels for treatment of root canals in different types and sizes of teeth in accordance with the invention.

TABLE 1
Preferred Power Levels for Various Tooth Types

| | Approx. Average | Range of Preferred |
|---|---|---|
| Tooth Type | Root Volume (μL) | Power Levels (watts) |
| Molar | 177 | 0.5 to 1.5 |
| Pre Molar | 88 | 0.5 to 1.0 |
| Cuspid | 67 | 0.5 to 0.75 |
| Laterals | 28 | 0.25 to 0.5 |
| Centrals | 28 | 0.25 to 0.5 |
| Lower | 28 | 0.1 to 0.25 |
| Centrals | | |

When the laser is immersed in the first solution, the laser is pulsed for a time preferably ranging from about 10 seconds to about 40 seconds, most preferably about 20 seconds. If the laser is pulsed for longer than about 40 seconds, excessive thermal energy can begin to develop in the fluid, potentially leading to deleterious heating effects in and around the tooth as described above. It has been found rather surprisingly that pulsing under the parameters of the invention causes a measurable temperature rise in the fluid medium of no more than a few degrees Celsius, if any, while still utterly destroying and/or disintegrating all nerve, pulp, and other tissue within the canal that also is observed to hydraulically self-eject from the canal during pulsing.

After the laser has been pulsed in the first solution, the first solution is allowed to stabilize and then laser pulsing treatment may be repeated again in the same or a different solution. In certain embodiments, the solution may be removed between repetitions of pulsing cycles of the laser to remove debris more gradually and to avoid any development or transfer of heat energy into the dentin surrounding wall or other adjacent structure. The coronal chamber and canal may be irrigated with a standard dental irrigant and solution may then be reinserted into the coronal chamber to perform an additional laser pulsing treatment. While any number of pulsing phases or cycles can be repeated, it is believed that a fully effective removal of all material within the canal can be achieved in less than about seven cycles.

To assist dentists in performing root canal treatments according to the present invention, a photoacoustic activity index has been developed which provides relationships between the various parameters, machine setting, and the like which have been found to be important in the practice of the inventive procedure. Factors which appear important in the practice of the invention include the power level, laser pulse frequency, the pulse duration, the proportion of average excitable functional groups per molecule in the first solution, the diameter of the laser optical fiber, the number of pulsing cycles repeated in completing an extirpation procedure, the duration of each cycle, the viscosity of the first solution, and the distance between the tip and the end of the cladding. Coefficients have been determined which relate deviations of certain of the above factors from what is believed to be the ideal or the most preferred factor value. Tables of these coefficients are shown below:

| | | Preferred | |
|---|---|---|---|
| | | Range of | |
| | Approx. Average | Power Levels | Power Density |
| Tooth Type | Root Volume (uL) | (watts) | Coefficient (DPD) |
| Molar | 177 | 0.5 to 1.5 | 1 |
| Pre Molar | 88 | 0.5 to 1.0 | 1 |
| Cuspid | 67 | 0.5 to 0.75 | 1 |
| Laterals | 28 | 0.25 to 0.5 | 1 |
| Centrals | 28 | 0.25 to 0.5 | 1 |
| Lower | 28 | 0.1 to 0.25 | 1 |
| Centrals | | | |

| Frequency | Pulses per |
|---|---|
| Coefficient | Second |
| C (fq) | (Value in HZ) |
| 0.4 | 2 HZ |
| 0.6 | 5 HZ |
| 0.9 | 10 HZ |
| 1 | 15 HZ |
| 0.5 | 20 HZ |
| 0.2 | 25 HZ |

| Pulse Duration | |
|---|---|
| Coefficient | Pulse Duration |
| C (pw) | Value in micro sec (μs) |
| 1 | <50 |
| 0.9 | 50 |
| 0.7 | 100 |
| 0.3 | 150 |
| 0.2 | 200 |
| 0.1 | 1000 |

| Hydroxyl | Average quantity of |
|---|---|
| Coefficient | excitable groups |
| C (hy) | per fluid molecule |
| 1 | >2 |
| 0.9 | 2 |
| 0.7 | 1 |
| 0.5 | Part or Mixture |
| 0 | none |

| Fiber | |
|---|---|
| Diameter | |
| Coefficient | Fiber Diameter |
| C (fd) | Value in microns |
| 0.8 | >400 |
| 1 | 400 |
| 0.8 | 320 |
| 0.5 | 200 |
| 0.3 | <200 |

| Repetition Cycle | |
|---|---|
| Coefficient | Repetition Cycles |
| C (rp) | (repetitions) |
| 0.3 | >7 |
| 0.5 | 6 |
| 0.7 | 5 |
| 1 | 4 |
| 0.9 | 3 |
| 0.6 | 2 |
| 0.3 | 1 |

| Cycle Duration | |
|---|---|
| Coefficient | Cycle Duration |
| C (sa) | (Value in seconds) |
| 0.2 | >40 |
| 0.6 | 40 |
| 0.9 | 30 |
| 1 | 20 |
| 0.5 | 10 |
| 0.2 | <10 |

| Viscosity | |
|---|---|
| Coefficient | Fluid Viscosity |
| C (vs) | (Centipoise) |
| 1 | <1 |
| 0.9 | 1 |
| 0.1 | >500 |
| 0.05 | >1000 |

| Cladding | Distance Between |
|---|---|
| Separation | Terminus of Cladding |
| Length | and Apex of Tip |
| Coefficient | Value in millimeters |
| C (sl) | (mm) |
| 0.4 | 2 |
| 0.6 | 3 |
| 0.9 | 4 |
| 1 | 5 |
| 0.9 | >5 |
| 0.3 | >10 |

A practitioner may input coefficients from the above tables correlating to equipment, setting, and material parameters into the following equation:

Photoacoustic Activity Index (“PA” Index)=DPD×C(fq)×C(pw)×C(hy)×C(fd)×C(rp)×C(sa)×C(vs)×C(sl)

If the resulting PA Index value is greater than about 0.1, more preferably above about 0.3, then the equipment and materials may generally be acceptable to produce an effective photoacoustic wave for disintegration and substantially complete and facile removal of all root canal nerve, pulp, and other tissue from within the canal. If the PA Index is below about 0.1, it may indicate a need to modify one's equipment setup, setting, and method parameters in order to more closely approach the desired PA index of 1 or unity.

Using the invention parameters and procedures, root canal tissue and other material to be removed or destroyed is not believed to be removed or destroyed via thermal vaporization, carbonization, or other thermal effect due primarily to exposure to high temperatures, but rather through a photoacoustic streaming of and other activities within liquids in the canal which are laser activated via photon initiated photoacoustic streaming (PIPS™). A photoacoustic wave with a relatively high leading edge is generated when the laser light transitions from the exposed surface of the fiber optic material into the solution. The laser light is believed to create very rapid and relatively intense oscillations of waves through the solution emanating from the interface of the exposed surface of the fiber optic and the surrounding liquid. The rapid, intense microfluctuations in the light energy emitted is believed to cause rapid excitation and/or expansion and de-excitation and/or expansion of hydroxyl-containing molecules adjacent the exposed surface of the fiber generating, among other things, photoacoustic waves of energy which propagates through and into the root canal system and oscillates within the system. These intense photoacoustic waves are believed to provide substantial vibrational energy, which expedites the breaking loose of and/or cell lysis and other effects to bring about a rapid and facile degradation/disintegration of substantially all tissue in the root canal and lateral canal systems immersed in the solution. The pulsing photoacoustic energy waves in combination with the chemistry of the fluid also is believed to cause intense physically disruptive cycling of expanding and contracting of nerve and other tissue which porositizes, expands, and ultimately disintegrates the nerve and other tissue in the canal without any significant thermally induced carbonization or other thermal effects of the same so that the resulting solution/mixture containing nerve and other tissue remains is observed to be self-ejected or basically “pumped” by a hydraulic effect out of the canal.

The photoacoustic effect creates energy waves that propagate throughout the fluid media in the main root canal and into the lateral canals, thereby cleaning the entire root system. These energy waves provide vibrational energy, which expedites the breaking loose of and/or causing cell lysis of the biotics and inorganics in the root canal and lateral canal systems. In addition these vibrational waves help the propagation of the fluids into and throughout the main and lateral canal systems. Radiant light energy can fuse the root canal wall surface making it impossible to clean and debride the small passages behind the fused areas. The use of a substantially incompressible fluid medium, on the other hand, causes the waves produced by the photoacoustic effect to be instantly transmitted through the lateral canals. Also, since the canals are tapered in a concave fashion, the photoacoustic wave is believed to be amplified as it transverses toward the end of the lateral canals for further intensification of the destruction towards apical or cul de sac areas.

In general, light travels in a straight line. However, in a fluid light can be bent and transmitted around corners, but this transmission is minimal compared to the straight-line transmissibility of light. A sonic or shock wave on the other hand is easily transmitted around corners and through passages in a fluid. For example, air is a fluid. If you stood in one room and shined a bright light from that room into a hallway that was at right angles to that room, the intensity of the light would decrease the farther you go down the hallway. If you then went into a room at the end of the hallway and went to a back corner of the room, the light might be very dim. However, if while standing at the same location as the light source, you yelled vocally at the hallway, you could most likely hear the sound in the back corner of the back room. This is because sound is propagated multidirectionally by the vibration of molecules instead of primarily in a straight line like light.

In certain embodiments of the invention, a second dissolution solution may be added to the canal after treatment with the energy source/first solution. This dissolution solution chemically dissolves and/or disintegrates any remaining nerve structure or other debris that may remain in the main canal or in any lateral canals. Preferred dissolution solutions include hypochlorite, sodium hypochlorite, perborate, calcium hydroxide, acetic acid/lubricant/doxycycline and other like nerve tissue or matrix dissolving substances such as chelating agents (EDTA) and inorganic agents such as titanium oxides.

Finally, after desired tissue has been removed from the tooth interior, the canal may be irrigated to remove any remaining debris and remaining solution, and then obturated with a material of choice, such as gutta percha, root canal resin, etc., according to standard practices in the industry.

In certain embodiments, various fluids may be used in conjunction with each other for various endodontic and root canal procedures. The following fluids are energetically activated by photoacoustic wave generation technology (PIPS) during their use throughout these examples. In a preferred embodiment, a first fluid including water and about 0.1% to about 20%, most preferably about 20%, urea hydrogen peroxide (weight/volume) containing about 0.01% to about 1% hexadecyl-trimethyl-ammonium bromide (cetrimide) is introduced into a tooth canal through an opening formed in the crown of a tooth. The first fluid is used to cause rapid nerve expansion so that any nerve tissue remaining in and adjacent the pulp chamber expands and is more easily removed from the pulp chamber. Preferably, a second fluid including water and about 0.1% to about 10%, most preferably about 5% hypochlorite (volume/volume) containing from about 0.01% to about 1% cetrimide is introduced into the tooth canal through the opening formed in the crown of the tooth. The second fluid is used to dissolve any remaining nerve tissue so that any nerve tissue remaining in and adjacent the pulp chamber is more easily removed by a fluid. Preferably, a third fluid including water and from about 0.1% to about 20%, more preferably from about 15% to about 17% EDTA 15 (weight/volume) containing from about 0.01% to about 1% cetrimide is introduced into the tooth canal through the opening formed in the crown of the tooth. The third fluid is used to help remove any remaining smear layer which typically contains, for example, organic material, odontoblastic processes, bacteria, and blood cells.

In a related embodiment, the first fluid, the second fluid, and the third fluid are used as described above, and then a fourth fluid is introduced into the sulcus near the tooth that has been treated followed serially by a fifth fluid. The fourth fluid includes water and from about 0.01% to 1% cetrimide and the fifth solution includes water and from about 0.01% to about 2%, most preferably about 0.2% chlorhexidine (weight/volume).

In another related embodiment, the first fluid, the second fluid, and the third fluid are used as described above, and then a mixture of a fourth fluid and a fifth fluid is introduced into the sulcus near the tooth that has been treated. The fourth fluid includes water and from about 1% to about 20%, most preferably about 20% urea peroxide (weight/volume) containing 0.01% to 1% cetrimide (wt/vol). The fifth fluid includes water and from about 0.1% to about 10%, most preferably about 1% hypochlorite (weight/volume). When the fourth fluid and the fifth fluid are mixed together and introduced into the sulcus near a treated tooth, a rapid expansive bubbling and bactericidal fluid mixture forms that is capable of destroying plaque and useful as a liquid defining a reservoir for a laser tip as described herein to be inserted and used as described herein.

In yet a further related embodiment, the first fluid, the second fluid, and the third fluid are used as described above, and then a mixture of a fourth fluid, a fifth fluid and a sixth fluid is introduced into the sulcus near the tooth that has been treated. The fourth fluid includes water and from about 1% to about 20%, most preferably about 20% urea peroxide (weight/volume) containing 0.01 to 1% cetrimide (wt/vol). The fifth fluid includes water and from about 0.1% to about 10%, most preferably about 1% hypochlorite (volume/volume). The sixth fluid includes water and from 0.01% to about 2%, most preferably about 0.2% chlorhexidine (weight/volume). When the fourth fluid, the fifth fluid and the sixth fluid are mixed together and introduced into the sulcus near a treated tooth, a rapid expansive bubbling and bactericidal fluid mixture forms that is capable of destroying plaque and useful as a liquid defining a reservoir for a laser tip as described herein to be inserted and used as described herein.

In yet another related embodiment, the first fluid, the second fluid, and the third fluid are used as described above, and then a mixture of a fourth fluid and a fifth fluid is introduced into the sulcus near the tooth that has been treated. The fourth fluid includes water and from about 0.1% to about 10%, most preferably about 1% sodium bicarbonate (weight/volume) buffered with sodium hydroxide to pH 9.6 to pH 11 containing 0.01% to 1% cetrimide, most preferably about pH 10. The fifth fluid includes water and from about 0.1% to about 10%, most preferably about 0.5% hypochlorite (weight/volume). When the fourth fluid and the fifth fluid are mixed together and introduced into the sulcus near a treated tooth, a rapid expansive bubbling and bactericidal fluid mixture forms that is capable of destroying plaque and useful as a liquid defining a reservoir for a laser tip as described herein to be inserted and used as described herein.

In yet a further related embodiment, the first fluid, the second fluid, and the third fluid are used as described above, and then a mixture of a fourth fluid, a fifth fluid and a sixth fluid is introduced into the sulcus near the tooth that has been treated. The fourth fluid includes water and from about 0.1% to about 10%, most preferably about 1% sodium bicarbonate (weight/volume) buffered with sodium hydroxide to pH 9.6 to pH 11 containing 0.01 to 1% cetrimide, most preferably about pH 10. The fifth fluid includes water and from about 0.1% to about 10%, most preferably about 1% hypochlorite (weight/volume). The sixth fluid includes water and from 0.01% to about 2%, most preferably about 0.2% chlorhexidine (weight/volume). When the fourth fluid, the fifth fluid and the sixth fluid are mixed together and introduced into the sulcus near a treated tooth, a rapid expansive bubbling and bactericidal fluid mixture forms that is capable of destroying plaque and useful as a liquid defining a reservoir for a laser tip as described herein to be inserted and used as described herein.

Preferably, after one or more treatment steps including use of a mixture of the fourth fluid and the fifth fluid, a mixture including EDTA to remove oxygen that may interfere with subsequent endodontic and/or periodontal treatment steps is rinsed in a tooth and/or a sulcus adjacent a tooth.

Qualitative experimentation was performed placing a fluid into a Dampen dish located on a Formica surface. The laser applicator tip was placed into the fluid and fired repetitively. The photoacoustic wave vibrated the Dampen dish on the Formica surface making an audible sound. For a specific tip this audible sound increased with an increasing power level of the laser. This was verified by placing a sound level meter one inch away from the Dampen dish and recording the dB level. This implies that the power level is proportional to the amplitude of the photoacoustic wave. Next, the laser power level was held constant and the tip was changed. The tapered tip and a tip with a stripped sheath produced a greater photoacoustic wave than the standard flat tip. A tapered, stripped tip was then frosted or etched. This tip was tested and showed a greater photoacoustic wave generated than the non-frosted version. This was verified to be true at three different power levels. It would appear that since the power level was held constant, the photoacoustic wave amplitude would also be proportional to the exposed area and the surface treatment.

In a quantitative investigation of the applicator tip a MEMS Pressure sensor was utilized to measure the photoacoustic wave amplitude. This testing has shown a dramatic increase in the photoacoustic wave propagation caused by changes in the geometry and texturing of the tip. The inventors have also discovered that stripping of the cladding from the end of the applicator tip results in increases in the photoacoustic wave effect. In this regard, a small plastic vial was fitted with a fluid connection that was close coupled hydraulically to a miniature MEMS piezo-resistive pressure sensor (Honeywell Model 24PCCFA6D). The sensor output was run through a differential amplifier and coupled to a digital Oscilloscope (Tektronics Model TDS 220). The vial and sensor were filled with water. Laser tips having varying applicator tip configurations were fully submerged below the fluid level in the vial and fired at a frequency of 10 HZ. The magnitude of the photoacoustic pressure waves was recorded by the pressure sensor.

A 170% increase in pressure measured from generation of the photoacoustic waves was observed for the tapered tip versus the standard blunt-ended tip. A 580% increase in pressure measured from generation of the photoacoustic wave was observed for textured (frosted) tapered tips versus the standard blunt-ended tip. Rather than emitting in a substantially linear direction, the frosting disperses the light omnidirectionally causing excitation and expansion of more fluid molecules.

An increase in photoacoustic wave generation was seen by stripping the polyamide sheath away from about 2 mm to about 10 mm from the tapered end. Although laser light is coherent and typically travels substantially in a straight line, some light bounces off of the polyamide sheath at an angle. As this light travels down the light path it continues bouncing off of the inside of the polyamide sheath and will eventually exit at an angle to the sheath once the sheath stops and exposes a non sheathed section. Therefore, some of the laser light would also exit where the polyamide sheath has been removed, upstream of the tapered tip end. A tip with the sheath removed for 2 to 10 mm directly upstream of the tapered section was placed in the above-mentioned test set up and showed markedly better production of photoacoustic waves.

In various other embodiments of the invention, energy sources other than lasers may be used to produce the photoacoustic waves including, but not limited to, other sources of light energy, sonic, ultrasonic, photo-acoustic, thermo-acoustic, micromechanical stirring, magnetic fields, electric fields, radio-frequency, and other exciter mechanisms or other similar forms that can impart energy to a solution. Some of these sources penetrate the tooth structure externally. Additional subablative energy sources may be used to create other types of pressure waves in a solution, such as chemoacoustic waves (shock waves created by rapid chemical expansion creating shock and pressure waves). Such waves can be created for example by loading the nanoparticles with a chemical that expands rapidly upon excitation, coating nanoparticles with a hard shell (e.g. polyvinyl alcohol), and activating the chemistry with an energy source such as optical, ultrasonic, radio-frequency, etc. As the activating chemical expands, pressure builds up in the hard shell, when the shell bursts it creates a shock wave that can propagate throughout the fluid similar to a photoacoustic wave. Additionally, a photoacoustic wave can be the activating energy source for producing the chemoacoustic wave.

Further, embodiments of the present invention may be used for various procedures other than root canal treatment, such as for treatment of dental caries, cavities or tooth decay. Additionally, the present invention may be usable for treatments of bone and other highly networked material where infection is problematic, e.g. dental implants, bone infection, periodontal disease, vascular clotting, organ stones, scar tissues, etc. Adding a tube structure around the tip which might be perforated and will allow introduction of a fluid around the tip that will allow the photoacoustic waves to be directed into more difficult areas that do not contain fluid volume such as periodontal and gum tissue. This would be considered a type of photoacoustic transmission tube.

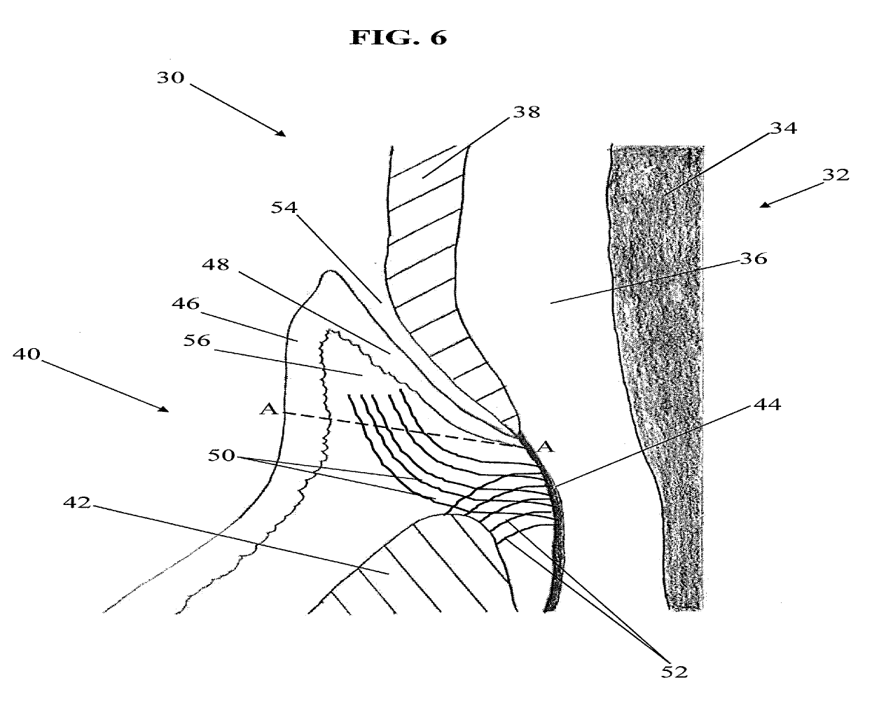

Certain periodontal treatment embodiments are contemplated including a method and apparatus for treating gingival and periodontal regions near a tooth structure. FIG. 6 shows a cutaway view of a tooth and gum interface region 30 including a portion of a tooth 32 including tooth pulp 34, tooth dentin 36, and tooth enamel 38; a portion of gum tissue 40 including a portion of an alveolar bone 42, cementum 44, oral epithelium 46, sulcular epithelium 48, dentogingival fibers 50, and dentoalveolar fibers 52; and a sulcus 54 defining the open region or “pocket” between the tooth 32 and a free dental gingival margin 56 of the gum tissue 40 located above the dashed line A-A. The term “sulcus” and “pocket” refer to the volume between one or more teeth and gingival tissue.

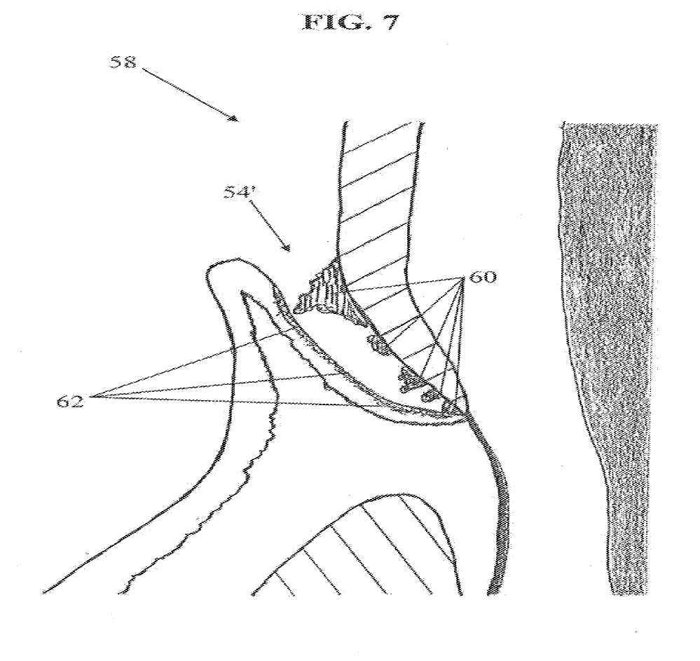

The sulcus 54 and surrounding area is a notorious place for plaque to develop. The sulcus 54 and surrounding area is also notorious area for calculus deposits to form. FIG. 7 shows a cutaway view of a tooth and gum interface region 58 including calculus deposits 60 and a diseased portion of a sulcular epithelium 62. Although plaque is relatively soft and may often be removed by routine brushing, calculus deposits often require significantly more force to remove, especially when such calculus deposits have attached to the cementum 44. A calculus deposit—commonly referred to as tartar—is a cement-like material that is often scraped off of teeth during a routine dental visit and followed up with some degree of chemical treatment including, for example, fluoride rinsing. Often, such scraping causes undesirable swelling of the teeth and gums, and healthy tissue including much needed cementum 44 is inadvertently removed along with the calculus deposits. The inadvertent removal of cementum 44 often results in less adhesion between teeth and gums, causing sagging of the gums. When the gum tissue 40 sags, additional surfaces of the tooth 32 are exposed, some of which may not protected by enamel 38. This is undesirable and can lead to deteriorating tooth and gum health.

Applicants have surprisingly found that the endodontic laser techniques including apparatuses and methods described herein are also applicable with respect to gingival and periodontal treatment. Such laser treatment is capable of disengaging and disintegrating plaque, destroying undesirable bacterial cells, and disengaging and disintegrating calculus deposits. It is believed that the photoacoustic waves emitted from the laser 10 cause, among other things, the lysing of bacterial cells.

In a first embodiment, an apparatus and method of treatment for treating mild to moderate periodontal disease is disclosed wherein mild to moderate periodontal disease is indicated by pockets having a depth of from about 4 mm to about 5 mm. The pulsing laser 10 including the optical fiber 14 with the applicator tip 20 is preferably used. The tip 20 preferably consists essentially of quartz.

The associated method includes the steps of (A) optionally and gently pulling the free dental gingival margin 56 from adjacent teeth to widen the sulcus 54, (B) introducing a fluid to the sulcus 54 to create a reservoir of fluid within the sulcus 54 (C) removing the diseased epithelial lining from the pocket using the laser 10 of a first type with the optical fiber 14 of a first size wherein the laser 10 is adjusted to a first setting, (D) removing calculus deposits from one or more teeth using the laser 10 of a second type with the optical fiber 14 of a second size wherein the laser 10 is adjusted to a second setting, (E) optionally removing any remaining calculus deposits using a piezo scalar, (F) modifying the dentin surface using the laser 10 with the optical fiber 14 of a third size wherein the laser 10 of a third type is adjusted to a third setting, and (G) inducing fibrin clotting at areas where treatment has occurred. If the treated tissue still looks diseased after treatment, follow-up treatment is to be commenced preferably about one week later using the laser with the optical fiber 14 of the first size wherein the laser 10 is adjusted to the first setting. Treatment is preferably initiated on the most diseased area of a mouth (i.e., the quadrant of a mouth having the deepest and most pockets).

In one preferred embodiment, steps (C) and (E) are not included. In other embodiments other steps may be left out or otherwise altered depending on a particular patient's needs or other reasons. In certain embodiments in the above or any other method disclosed herein, a single type of laser may be used for multiple or even all of the steps, although, as disclosed, different types of lasers may be preferable for certain steps.

If the first laser type is Nd doped (e.g., Nd:YAG), the first size preferably ranges from about 300 microns to about 600 microns in diameter and the first setting includes a pulse width of from about 100 μs to about 700 μs (preferably about 100 μs) and a power setting of about 2.0 to about 4.0 watts (W). If the first laser type is a Diode laser (about 810 to about 1064 nanometers (nm)), the first size preferably ranges from about 300 microns to about 1000 microns in diameter and the first setting includes a continuous wave setting and a power setting of from about 0.2 W to about 4.0 W.

If the second laser type is Er doped, the second size preferably ranges from about 400 microns to about 1000 microns in diameter, and the second setting preferably includes a pulse width of from about 50 μs to about 300 μs, an energy setting of from about 10 mJ to about 100 mJ, and a frequency of from about 2 Hz to about 25 Hz. If the second laser type is Er, Cr doped, the second size preferably ranges from about 600 microns to about 1000 microns in diameter, the second setting preferably includes a pulse width of from about 50 μs to about 100 μs, an energy amount of from about 10 mJ to about 100 mJ, and a frequency of from about 2 Hz to about 50 Hz.

If the third laser type is Er doped, the third size preferably ranges from about 400 microns to about 1000 microns in diameter, and the third setting preferably includes a pulse width of from about 50 μs to about 300 μs, an energy setting of from about 10 mJ to about 100 mJ, and a frequency of from about 2 Hz to about 50 Hz.

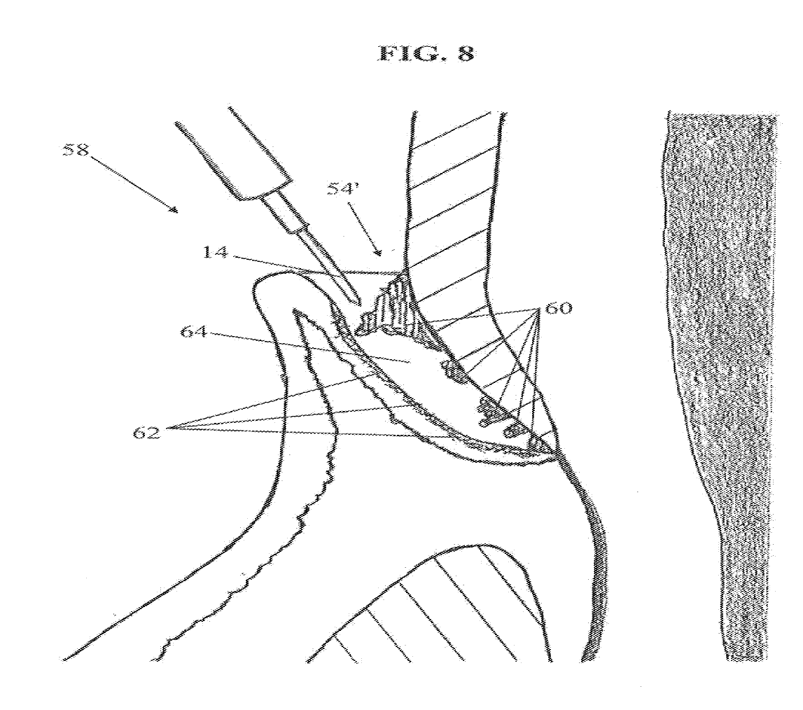

In its simplest form step (B) uses water. FIG. 8 shows a sulcus 54′ filled with a fluid, defining a reservoir 64 for periodontal treatment using photoacoustic technology. Step (C) preferably includes removing the epithelial lining by moving the applicator tip 20 in a side to side sweeping motion starting at or near the top of the sulcus 54 and slowly moving to a location of about 1 mm from the base of the sulcus 54 where the sulcular epithelium 48 and the cementum 44 attach (assuming these structures are still attached) as shown in FIG. 8. Step (C) should preferably take from about 10 seconds to about 15 seconds to perform. In step (C), if the laser type is Nd doped, the first size of the light fiber 14 is preferably about 320 microns and the first setting of the laser 10 preferably includes a pulse width of about 100 μs VSP, a frequency of about 20 Hz, and a power setting of from about 2.0 W to about 3.0 W.

Step (B) preferably includes using the fourth fluid and the fifth fluid described above (i.e., the fourth fluid including water and from about 0.5% to about 20%, most preferably about 2% urea peroxide containing 0.01 to 1% hexadecyl-trimethyl-ammonium bromide (cetrimide), and the fifth fluid including water and from about 0.0125% to about 5.0%, most preferably about 0.25% hypochlorite). These fluids are added serially, whereby the fourth solution is added first and activated individually by photoacoustic wave generation technology, followed shortly by addition of the second solution which is then itself activated by photoacoustic wave generation technology. Alternatively, these fluids are mixed together just prior to use and are then activated by photoacoustic wave generation technology.

In a related embodiment, step (B) preferably includes using the fourth fluid and the fifth fluid described above (i.e., the fourth fluid including water and from about 0.5% to about 20%, most preferably about 2% urea peroxide containing 0.01 to 1% hexadecyl-trimethyl-ammonium bromide (cetrimide), and the fifth fluid including water and from about 0.0125% to about 5.0%, most preferably about 0.25% hypochlorite), followed by a sixth fluid including water and from about 0.01% to about 2%, most preferably about 0.2% chlorhexidine (weight/volume).

In another related embodiment, step (B) includes using the a fourth and fifth fluid that includes water and from about 0.1% to about 10%, most preferably about 1% sodium bicarbonate (weight/volume) buffered with sodium hydroxide to pH 9.6 to pH 11 containing 0.01 to 1% cetrimide, most preferably about pH 10. The fifth fluid includes water and from about 0.1% to about 10%, most preferably about 1% hypochlorite (weight/volume).

In yet a further related embodiment, step (B) includes using a mixture including a seventh fluid, an eighth fluid and a ninth fluid. The fluid mixture is introduced into the sulcus near the tooth that has been treated. The seventh fluid preferably includes water and from about 0.1% to about 10% and most preferably about 1% sodium bicarbonate (weight/volume) buffered with sodium hydroxide to a pH value ranging from about 9.6 to about 11 (preferably about 10) wherein the sodium hydroxide preferably includes from about 0.01% to about 1% cetrimide. The eighth fluid includes water and from about 0.1% to about 10% (most preferably about 1%) hypochlorite (weight/volume). The ninth fluid includes water and from about 0.01% to about 2% (most preferably about 0.2%) chlorhexidine (weight/volume).

Preferably, for step (D) and other steps described herein wherein the applicator tip is inserted into a sulcus and photoacoustic wave generation technology is used to create photoacoustic waves, an appropriately dimensioned laser applicator tip 20 is preferably placed into the sulcus until it is at least fully immersed in the solution therein. By “fully immersed” it is meant liquid level is even with the edge of the cladding or other covering on the optical fiber 18. Preferably, the distal most edge of any cladding or covering 18 on the optical fiber 18 adjacent the tip is spaced from about 1 mm to about 10 mm from the distal end of the distal end tip or end of the optical fiber, most preferably about 3 mm therefrom. As a result, up to about 10 mm and most preferably about 3 mm of the distal end of the optical fiber is uncovered. In other embodiments, however, the distal most edge of any cladding or covering 18 on the optical fiber adjacent the tip is substantially at the distal end of the distal end tip or end of the optical fiber. Preferably, all or substantially all of the length of this uncovered part of the tip end is immersed. If the uncovered part of the applicator tip is not fully immersed, sufficient energy may not be transferred to the fluid in the sulcus since light will be permitted to escape to the environment above the liquid surface. Accordingly, it is believed that spacing the distal-most or outermost end edge of the cladding more than about 10 mm should be avoided, as that can diminish the effectiveness of the system.

In some applications, it may be necessary to provide a dam and reservoir around and above the opening in the tooth in order to increase the volume and level of fluid available for immersion of the uncovered area of the end of the optical fiber. The larger liquid volume and deeper immersion of the uncovered area of the tip end is believed to enable application of sufficient energy levels to produce the desired photoacoustic wave intensity in such instances. Such instances may include, for example, smaller pockets where treatment is desired. In certain applications where a dam or reservoir is used, particularly in veterinary applications for larger animals, it may be desirable to use a laser tip with more than 20 mm of space between the tip end and the cladding due to the larger volume of fluid.

Preferably, for step (D) and other steps described herein wherein the applicator tip is inserted into a sulcus and photoacoustic wave generation technology is used, the various embodiments of fluids described with respect to Step (B) are also preferably used in Step (D).

Step (D) preferably includes removing calculus deposits by moving the applicator tip 20 in a substantially side to side sweeping motion starting at or near the top of the sulcus 54 and slowly moving down the tooth 32 in contact therewith (preferably using a light touch), pausing on any calculus deposits to allow the laser 10 to remove the deposit(s). Step (D) may include multiple repetitions, often from about 3 to about 6, to ensure most of the calculus deposits have been removed from the tooth 32 or cementum 44 surfaces. In step (D), the second size of the optical fiber 14 is preferably about 600 microns in diameter. The second setting of the laser 10 preferably includes a pulse width of about 100 μs VSP and a frequency of about 15 Hz.

Hand tools should only be used in step (E) as a last resort because such tools often remove much needed cementum 44 from the tooth 32. In some embodiments, Step (F) uses substantially the same techniques, sizes, and settings as step (C). During Step (F), the applicator tip 20 is preferably held substantially parallel to the length of the tooth 32 while being in contact with the tooth 32. Step (F) should take from about 5 to about 15 seconds depending on the depth of the sulcus 54. During any follow-up treatment, pressure should be placed on all lased areas for about 3 minutes to better ensure fibrin clotting.

Step (G) preferably includes treating all pockets having a depth of 5 mm or more if, for example, tissue inflammation or bleeding persists. Treatment during Step (G) is similar to the technique used during Step (C). However, for typical adult human patients, the treatment depth is restricted to moving no more than about 3 mm into a sulcus so as to avoid disturbing healing tissues below such depth. The treatment action occurring in Step (G) has the effect of removing remaining diseased tissue while biostimulating surrounding sulcular tissue.

In a second embodiment, an apparatus and method of treatment for advanced periodontal disease is disclosed wherein advanced periodontal disease for typical adult human patients is indicated by pockets having a depth of from about 6 mm to about 10 mm or more. The pulsing laser 10 including the optical fiber 14 with the applicator tip 20 is preferably used. The associated method preferably includes the steps of (A)′ gross scaling a treatment site (e.g., a quadrant of teeth and surrounding tissue) with a piezo scaler, avoiding the use of hand instruments in the cementum if possible; (B)′ introducing a fluid to a sulcus to create a reservoir of fluid within the sulcus; (C)′ removing the diseased epithelial lining located in an upper portion of the pocket using the laser 10 of a fourth type with the optical fiber 14 of a fourth size wherein the laser 10 is adjusted to a fourth setting; (D)′ removing calculus deposits from one or more teeth using the laser 10 of a fifth type with the optical fiber 14 of a fifth size wherein the laser 10 is adjusted to a fifth setting; (E)′ removing any remaining calculus deposits using a piezo scaler; (F)′ remove diseased epithelial lining to the bottom of the sulcus using the laser of a sixth type with the optical fiber of a sixth size wherein the laser 10 is adjusted to a sixth setting; (G)′ modifying the dentin surface including removal of calculus using the laser 10 of a seventh type with the optical fiber 14 of a seventh size wherein the laser 10 is adjusted to a seventh setting; (H)′ removing the diseased epithelial lining located in a lower portion of the sulcus using the laser 10 of an eighth type with the optical fiber 14 of an eighth size wherein the laser 10 is adjusted to an eighth setting; (I)′ dissecting proximal periodontal attachment with bone using the laser 10 of a ninth type with the optical fiber 14 of a ninth size wherein the laser 10 is adjusted to a ninth setting; (J)′ penetrating the cortical plate of adjacent bone tissue with an endodontic explorer to accomplish cortication of any bony defect; (K)′ inducing fibrin clotting using the laser 10 of a tenth type with the optical fiber 14 of a tenth size wherein the laser 10 is adjusted to a tenth setting; and (L)′ placing one or more barricades or periacryl on all treated areas to prevent fibrin clots from washing out. Optionally, an enzyme inhibition mixture may be added to any collagen plug resulting from fibrin clotting in this or any other similar embodiment described herein. This optional step would extend the life of any applicable fibrin clot which, in turn, would promote decreased epithelial movement into the sulcus which would enhance tissue regeneration.

Treatment is preferably initiated on the most diseased area of a mouth (i.e., the quadrant of a mouth having the deepest and most pockets). If more than two quadrants of a mouth require treatment, the most diseased two quadrants should be treated first, followed up by treatment of the remaining quadrant(s) about one week later.

In one preferred embodiment, steps (C)′, (H)′ and (K)′ are not included. In another preferred embodiment, steps (F)′, (I)′ and (J)′ are not included. In another embodiment, steps (G)′ and (H)′ a performed in reverse order. In yet another embodiment, steps (K)′ and (L)′ are performed in reverse order. In other embodiments other steps may be left out, added, or otherwise altered depending on many factors including without limitation a particular patient's needs, availability of supplies, availability of laser technology, and other reasons.

If the fourth laser type is Nd doped (e.g., Nd:YAG), the fourth size preferably ranges from about 300 microns to about 600 microns in diameter and the fourth setting includes a pulse width of from about 100 μs (VSP) and a power setting of about 0.2 to about 4.0 W. If the fourth laser type is Er or Er,Cr doped, the fourth size preferably ranges from about 400 microns to about 1000 microns in diameter, the fourth setting preferably includes a pulse width of from about 50 μs to about 300 μs, an energy amount of from about 10 mJ to about 100 mJ, and a frequency of from about 2 Hz to about 50 Hz. If the fourth laser type is a Diode laser (about 810 nm to about 1064 nm), the fourth size preferably ranges from about 300 microns to about 1000 microns in diameter and the fourth setting preferably includes a continuous wave setting and a power setting of from about 0.4 W to about 4.0 W.

If the fifth laser type is Er doped, the fifth size preferably ranges from about 400 microns to about 1000 microns in diameter, the fifth setting preferably includes a pulse width of from about 50 μs to about 300 μs (SSP), an energy amount of from about 10 mJ to about 100 mJ (more preferably from about 20 mJ to about 40 mJ), and a frequency of from about 2 Hz to about 50 Hz (more preferably about 15 Hz to about 50 Hz). If the fifth laser type is Er or Er,Cr doped, the fifth size preferably ranges from about 400 microns to about 1200 microns in diameter, the fifth setting preferably includes a pulse width of from about 50 μs to about 300 μs, an energy amount of from about 10 mJ to about 200 mJ, and a frequency of from about 2 Hz to about 50 Hz.

If the sixth laser type is Er or Er,Cr doped, the sixth size preferably ranges from about 400 microns to about 1000 microns in diameter, the sixth setting preferably includes a pulse width of from about 50 μs to about 300 μs, an energy amount of from about 10 mJ to about 100 mJ, and a frequency of from about 2 Hz to about 50 Hz. If the sixth laser type is a Diode laser (about 810 nm to about 1064 nm), the sixth size preferably ranges from about 300 microns to about 1000 microns in diameter and the sixth setting preferably includes a continuous wave setting and a power setting of from about 0.4 W to about 4.0 W.

If the seventh laser type is Er doped, the seventh size preferably ranges from about 600 microns to about 1000 microns in diameter, the seventh setting preferably includes a pulse width of from about 50 μs to about 100 μs, an energy amount of from about 10 mJ to about 100 mJ, and a frequency of from about 2 Hz to about 50 Hz. If the seventh laser type is Er or Er,Cr doped, the seventh size preferably ranges from about 400 microns to about 1000 microns in diameter, the seventh setting preferably includes a pulse width of from about 50 μs to about 300 μs, an energy amount of from about 10 mJ to about 200 mJ, and a frequency of from about 2 Hz to about 50 Hz. If the eighth laser type is Er doped, the eighth size preferably ranges from about 600 microns to about 1000 microns in diameter, the eighth setting preferably includes a pulse width of from about 50 μs to about 100 μs, an energy amount of from about 10 mJ to about 100 mJ, and a frequency of from about 2 Hz to about 50 Hz. If the eighth laser type is Er or Er,Cr doped, the eighth size preferably ranges from about 400 microns to about 1000 microns in diameter, the eighth setting preferably includes a pulse width of from about 50 μs to about 300 μs, an energy amount of from about 10 mJ to about 200 mJ, and a frequency of from about 2 Hz to about 50 Hz.

If the ninth laser type is Er doped, the ninth size preferably ranges from about 600 microns to about 1000 microns in diameter, the ninth setting preferably includes a pulse width of from about 50 μs to about 100 μs, an energy amount of from about 10 mJ to about 100 mJ, and a frequency of from about 2 Hz to about 50 Hz. If the ninth laser type is Er or Er,Cr doped, the ninth size preferably ranges from about 400 microns to about 1000 microns in diameter, the ninth setting preferably includes a pulse width of from about 50 μs to about 600 μs, an energy amount of from about 10 mJ to about 200 mJ, and a frequency of from about 2 Hz to about 50 Hz.

If the tenth laser type is Nd doped (e.g., Nd:YAG), the tenth size preferably ranges from about 300 microns to about 350 microns in diameter (more preferably about 320 microns) and the tenth setting includes a pulse width of from about 600 μs to 700 μs (LP) (more preferably about 650 μs), a frequency of from about 15 Hz to about 20 Hz, and a power setting of about 3.0 to about 4.0 W. Clotting may also be induced by use of an Er-YAG laser by decreasing the power of the laser by increasing the pulse width to a range of from about 100 μs to about 600 μs to increase interaction with tooth root surfaces. Alternatively, laser power may be decreased by using an adapter (e.g., a filter) between a laser source and the zone where the laser is applied to a patient or other subject in order to attenuate laser signal. The option of using an Er doped laser is also available for fibrin clotting steps described in other embodiments herein.

Step (B)′ preferably includes using the fourth fluid and the fifth fluid described above (i.e., the fourth fluid including water and from about 0.1% to about 20%, most preferably about 10% urea peroxide, and the fifth fluid including water and from about 0.1% to about 10%, most preferably about 0.5% hypochlorite).

Step (C)′ preferably includes removing some of the epithelial lining by moving the applicator tip 20 in a side to side sweeping motion starting at or near the top of the sulcus and slowly moving down about 3 mm to about 5 mm. Step (C)′ should preferably take from about 10 to about 15 seconds to perform.

Step (D)′ preferably includes removing calculus deposits by moving the applicator tip 20 in a substantially side to side sweeping motion starting at or near the top of the sulcus and slowly moving down a tooth adjacent the sulcus, the tip preferably remaining in substantially continuous contact with the tooth, pausing proximate any calculus deposits to allow the laser 10 to remove the deposit(s). Such pauses may last from about 5 seconds to about 30 seconds. The method described herein is particularly well-suited for periodontic treatment because it leaves cementum substantially intact. Step (D)′ may include multiple repetitions, often from about 3 to about 6, to ensure most of the calculus deposits have been removed from the tooth or cementum surfaces. This technique should remove most calculus, bacteria, and endotoxins leaving the cementum mostly undamaged resulting in a desirable surface for reattachment of soft tissue to cementum.

Hand tools should only be used in step (E)′ as a last resort because such tools often remove much needed cementum from the tooth.

In a first embodiment, during Step (F)′, the applicator tip 20 is kept in substantially continuous contact with soft tissue surrounding the sulcus, starting at or near the top of the sulcus. The applicator tip 20 is moved in a sweeping motion (preferably a substantially side-by-side motion) toward the bottom of the sulcus. This step should take from about 10 to about 20 seconds to complete. The applicator tip 20 should not be kept at or near the bottom of the sulcus for more than about 3 to about 5 seconds to avoid compromising periodontal attachment. In a related embodiment of Step (F)′ in which the laser 10 is of the Diode type and the same general motion described above is used, the applicator tip 20 is extended to about 1 mm short of the sulcus depth because the laser 10 in this embodiment includes an end cutting fiber that cuts approximately 1 mm from the tip of the applicator tip 20.

In one embodiment of Step (G)′, the applicator tip 20 is preferably held substantially parallel to the length of a tooth while preferably remaining substantially in contact with such tooth. Step (G)′ should take from about 5 to about 15 seconds to complete depending on the depth of the sulcus. As an example, the same general motion as described with respect to Step (C)′ may be used in Step (G)′. In one embodiment, Step (G)′ further includes placing a stripped radial applicator tip into the sulcus to use photoacoustic wave generation technology for a period of from about 15 to about 25 seconds to accomplish substantially complete bacterial ablation prior to modifying the dentin surface.

Step (H)′ preferably includes removing some of the epithelial lining near the base of the sulcus by moving the applicator tip 20 in a side to side sweeping motion starting at or near the top of the sulcus. Step (H)′ should preferably take from about 10 to about 20 seconds to perform. A user should not spend more than about 5 seconds (and preferably no more than 3 seconds) at the base of the sulcus where the sulcular epithelium and the cementum attach (assuming these structures are still attached) in order to avoid compromising periodontal attachment.

Step (I)′ includes using photoacoustic wave generating technology as used in the previous step, starting at or near the bottom of the sulcus, to dissect fibrous periodontal attachment to a bony defect structure. Care should be taken to avoid disturbing the attachment of such fibers to bone on either side of a bony defect structure.

Step (J)′ includes using an endodontic explorer such as, for example, a double ended explorer available from DENTSPLY Tulsa Dental Specialties of Tulsa, Okla., to penetrate about 1 mm or more into an adjacent cortical plate. This penetration is preferably repeated from about 5 to about 15 times. This action allows for regenerative factors from the adjacent bone to be released which is necessary for bone regeneration. These penetrations also allow for angiogenesis which brings blood to the site quicker, giving a subsequent blood clot the nutrients needed to produce bone at a quicker rate.

Step (K)′ includes inducing fibrin clotting for bone generation by inserting the fiber 14 to a location about 75% of the depth of the sulcus and moving the applicator tip 20 in a substantially circular or oval-like motion throughout the sulcus, slowly drawing out gingivo-dental fibers. This will initiate fibrin clotting at or near the base of the sulcus. Step (K)′ may take from about 15 seconds to about 30 seconds to complete. The pocket being treated is preferably filled with blood prior to beginning Step (K); otherwise, it will be more difficult to obtain a good gelatinous clot. In a related embodiment, Step (K)′ includes inserting the applicator tip 20 to the depth of the sulcus that is along one side of the bony defect; activating the laser 10; moving the applicator tip 20 in a “J” shaped motion to draw out the fiber for a period of about 2 seconds; and proceeding through the defect for about 2 mm to about 3 mm in order to initiate a fibrin clot.

In one embodiment, Step (L)′ preferably includes placing one or more barricades and/or periacryl on one or more (preferably all) area treated using the laser 10 in order to prevent clots from washing out. Surgical dressings are preferably placed around one or more teeth and interproximal, and such dressings are preferably kept in place for about 10 days to prevent clots from washing out and to aid maturation of the treated bone and tissue. In a related embodiment, Step (L)′ includes placing an absorbable collagen sponge matrix in most and preferably all surgical sites to initiate clotting. This step protects the defect from, for example, bacterial invasion and provides a matrix for both hard and soft tissue regeneration. Blood platelets will aggregate near the collagen and the platelets will degranulate resulting in the release of coagulation factors which will combine with plasma to form a stable fibrin clot. This will step will, in certain embodiments, provide a matrix for bone regeneration and pocket elimination.

In addition to the steps listed above, an additional step preferably includes using chlorohexidine after the above-listed steps are completed. Preferably, the chlorohexidine is used no sooner than 48 hours after completion of the above-listed procedure, after which point the chlorohexidine is preferably used twice daily.

In addition to the periodontal embodiments described above, this application process may also be used in other soft tissue applications where it is necessary to expand the diseased tissue or material to allow more rapid access and penetration to healing agents, chemicals or biologicals; i.e. antibiotics, peptides, proteins, enzymes, catalysts, genetics (DNA, mRNA or RNA or derivatives) or antibody based therapeutics or combinations thereof. In some cases, the present methodology may be used to rapidly dissolve or destroy diseased tissue areas. Additionally, the present invention may be used to expand diseased tissue in an abscess, allowing for extremely rapid and efficient penetration of healing or biological agents. The porosity created in the tissue by photoacoustic waves may allow for rapid infusion with the subsequent chemical species that can impose destruction, healing or cleaning or a combination of these events. The speed of this healing action may allow medical procedures that currently are not viable because of extensive time required for standard healing processes, i.e., sometimes adjacent tissue is infected because the original infection cannot be controlled more rapidly than the infection propagates. In this case, expanding the diseased tissue to enhance porosity may allow near instantaneous access for the medication, e.g., antibiotic or other agents.

Furthermore, the present invention may be applied to begin, construct or stage the activation of cells and/or tissues, including the area of transplantation and use in stem or primordial cells accentuation, their attachment and/or stimulation for growth and differentiation. The present invention is also believed to be usable to activate cells, e.g., progenitor, primordial or stem cells, to promote inherent nascent bone or tissue growth and differentiation, as well as in transplantation where stem or primordial cells are accentuated in their attachment and stimulated for growth and differentiation.

In one of the alternate embodiments of this invention, nanotubes or other micro-structures can be moved around in a therapeutic fluid by applying a magnetic field. An alternating or pulsed magnetic field could impart significant motion and stirring of the therapeutic fluid. Since the field would penetrate the entire tooth, the stirring action would also occur throughout the lateral or accessory canal system. These moving micro-particles would also act as an abrasive on any bacteria, virus, nerve material, or debris within the canal system. The effect would be a more thorough circulation of the fluid throughout the canal system to provide superior cleaning and debridement of the canal system. Magnetic material can also be inserted into, adsorbed onto, or absorbed into the nanotube or other microstructure increasing its magnetic moment.

TiO₂ or other similar compounds can be activated and made bactericidal by exposing them to UV light or by inserting them in an electric field. Once excited these can destroy bacteria and other organic compounds such as remaining nerve tissue. Such compounds can be part of a therapeutic and can be activated by a UV light source pointed toward the therapeutic fluid, a UV source dipped into the fluid, or a UV laser source. These TiO₂ or other similar compounds can also be activated by an alternating or pulsed electric field. One means to supply such an electric field could be by an external device that would bridge the tooth. Since the field propagates throughout the entire tooth it would also react TiO₂ or other similar compounds within the accessory or lateral canals. This action could also be combined with the micro-particle based motion action mentioned above. This combination would more thoroughly clean and debride the canals. Since electric fields are generated externally and penetrate the entire root structure they could be used several months or on a yearly basis after the tooth is sealed to reactivate the titanium oxide and its bactericidal properties.

The foregoing description of preferred embodiments for this disclosure has been presented for purposes of illustration and description. The disclosure is not intended to be exhaustive or to limit the various embodiments to the precise form disclosed. Other modifications or variations are possible in light of the above teachings. The embodiments are chosen and described in an effort to provide the best illustrations of the principles of the underlying concepts and their practical application, and to thereby enable one of ordinary skill in the art to utilize the various embodiments with various modifications as are suited to the particular use contemplated. All such modifications and variations are within the scope of the disclosure as determined by the appended claims when interpreted in accordance with the breadth to which they are fairly, legally, and equitably entitled.

What is claimed is:

1. A method for treating a treatment zone including one or more teeth and tissue adjacent such tooth or teeth, the combination thereof defining a treatment pocket there between, the method comprising the steps of:
A. providing a laser system containing a source of a laser light beam and an elongate optical fiber connected to said source and configured to transmit said laser light beam to a tip thereof,
B. immersing at least a portion of the tip into a fluid reservoir located in the treatment pocket, the fluid reservoir holding a first fluid;
C. pulsing the laser light source at a first setting such that at least a substantial portion of any contaminants located in or adjacent the treatment pocket are destroyed or otherwise disintegrated into fragmented material in admixture in and with the first fluid, thereby forming a first fluid mixture, wherein the destruction or disintegration of a substantial portion of any contaminants located in or adjacent the treatment pocket using the laser light source is accomplished without generation of significant heat in the first fluid or associated mixture so as to avoid elevating the temperature of any gum, tooth, or other adjacent tissue more than about 5° C.

2. The method of claim 1 wherein the first setting of step (C) comprises an energy level of from about 2.0 W to about 4.0 W, a pulse width of from about 50 μs to about 300 μs, and a pulse frequency of from about 2 Hz to about 50 Hz.

3. The method of claim 1 wherein the first setting of step (C) comprises a power level of from about 10 mJ to about 100 mJ, a pulse width of from about 50 μs to about 300 μs, and a pulse frequency of from about 2 Hz to about 50 Hz.

4. The method of claim 1 wherein step (B) further comprises the step of introducing the first fluid into the treatment pocket in an amount sufficient to provide a fluid reservoir and step (C) further comprises removing substantially all of the first fluid mixture from the treatment pocket.

5. The method of claim 1 wherein step (C) further comprises destroying or otherwise disintegrating a substantial portion of any contaminants located in or adjacent the treatment pocket using the laser without generation of significant heat in the first fluid so as to avoid elevating the temperature of any gum, tooth, or other adjacent tissue more than about 3° C.

6. The method of claim 1 wherein step (C) further comprises the substeps of:
(1) removing calculus deposits in or proximate the treatment pocket by pulsing the laser light source at an energy level of from about 10 mJ to about 100 mJ and at a pulse width of from about 50 μs to about 300 μs, at a pulse frequency of from about 2 Hz to about 50 Hz, and moving the optical fiber tip in a first pattern, wherein the optical fiber has a diameter of from about 400 microns to about 1000 microns, and wherein a substantial portion of any calculus deposits located in or proximate the treatment pocket are disintegrated into fragmented material in admixture in and with the first fluid mixture, thereby forming a second fluid mixture; and
(2) optionally repeating step (C)(1) up to about six repetitions to remove substantially all calculus deposits from the treatment pocket.

7. The method of claim 1 wherein step (C) further comprises the substeps of:
(1) removing at least a portion of an epithelial layer of the treatment zone by pulsing the laser light source at the first setting wherein the first setting comprises settings selected from the group consisting of:
(a) a power level of from about 10 mJ to about 200 mJ, a pulse width of from about 50 μs to about 300 μs, and a pulse frequency of from about 2 Hz to about 50 Hz,
(b) an energy level of from about 2.0 W to about 4.0 W, a pulse width of from about 50 μs to about 300 μs, and a frequency of from about 15 Hz to about 50 Hz, and
(c) an energy level of from about 0.4 W to about 4.0 W and a continuous wave setting, and
moving the optical fiber tip in a first pattern, and wherein a substantial portion of any diseased epithelial tissue located in or adjacent the epithelial layer are destroyed or otherwise disintegrated into fragmented material in admixture in and with the first fluid mixture, thereby forming a second fluid mixture;
(2) removing calculus deposits in or proximate the treatment pocket by pulsing the laser light source at an energy level of from about 10 mJ to about 100 mJ and at a pulse width of from about 50 μs to about 300 μs, at a pulse frequency of from about 2 Hz to about 50 Hz, and moving the optical fiber tip in a second pattern, and wherein a substantial portion of any calculus deposits located in or proximate the treatment pocket are disintegrated into fragmented material in admixture in and with the second fluid mixture, thereby forming a third fluid mixture; and
(3) optionally repeating step (C)(2) up to about six repetitions to remove substantially all calculus deposits from the treatment pocket.

8. The method of claim 6 wherein step (C) further comprises the substep of:
(3) modifying the surface of dentin proximate the treatment pocket by pulsing the light beam producing apparatus at a energy level of from about 0.2 W to about 4 W, a pulse width of from about 50 μs to about 300 μs, and a pulse frequency of from about 2 Hz to about 50 Hz, and moving the optical fiber tip in a second pattern, and wherein the tip substantially remains in contact with the tooth during pulsing and wherein the tip is maintained substantially parallel to a root of an adjacent tooth during pulsing.

9. The method of claim 8 further comprising step (C)(4) including removing remaining diseased epithelial lining to a point substantially at the base of the pocket prior to modifying the surface of the dentin by pulsing the light beam producing apparatus at the first setting wherein the first setting comprises settings selected from the group consisting of:
(a) a power level of from about 10 mJ to about 100 mJ, a pulse width of from about 50 μs to about 300 μs, and a pulse frequency of from about 2 Hz to about 50 Hz; and
(b) an energy level of from about 0.2 W to about 4.0 W and a continuous wave setting.

10. The method of claim 8 further comprising step (C)(4) including removing substantially all remaining diseased epithelial lining to a point substantially at the base of the pocket by pulsing the laser light source at an energy level of from about 0.2 W to about 4.0 W, a pulse width of from about 50 μs to about 300 μs, and a pulse frequency of from about 2 Hz to about 50 Hz.

11. The method of claim 10 wherein step (C)(4) occurs before step (C)(3).

12. The method of claim 11 further comprising the step of:
(D) dissecting fibrous attachment between bone tissue and periodontal tissue along a bony defect at the base of the pocket by pulsing the laser light source at an energy level of from about 0.2 W to about 4.0 W, a pulse width of from about 50 μs to about 600 μs, and a pulse frequency of from about 2 Hz to about 50 Hz.

13. The method of claim 12 further comprising the step of:
(E) penetrating the cortical tissue of the bony defect adjacent the pocket to a depth of about 1 mm into the cortical tissue to form one or more perforations.

14. The method of claim 10 further comprising the step of:
(D) inducing a fibrin clot by inserting the optical fiber tip to about 75% the depth of the pocket, pulsing the laser light source at an energy level of from about 3.0 W to about 4.0 W, a pulse width of from about 600 μs to about 700 μs (LP), and a pulse frequency of from about 15 Hz to about 20 Hz, and wherein the optical fiber has a diameter of from about 300 microns to about 350 microns, and, for a period of about 15 seconds to about 30 seconds, moving the optical fiber tip in a curved motion while slowly drawing out the optical fiber.

15. The method of claim 13 further comprising the step of:
(F) inducing a fibrin clot by inserting the optical fiber tip to about 75% the depth of the 125 pocket, pulsing the light beam producing apparatus at an energy level of from about 3.0 W to about 4.0 W, a pulse width of from about 600 μs to about 700 μs (LP), and a pulse frequency of from about 15 Hz to about 20 Hz, and wherein the optical fiber has a diameter of from about 300 microns to about 350 microns, and, for a period of about 15 seconds to about 30 seconds, moving the optical fiber tip 130 in a curved motion while slowly drawing out the optical fiber tip.

16. The method of claim 14 further comprising the step of:
(E) placing a stabilizing treatment structure substantially on one or more locations treated by the laser light source.

17. The method of claim 15 further comprising the step of:
(G) placing a stabilizing treatment structure substantially on one or more locations treated by the laser light source.

18. The method of claim 6, wherein the first fluid mixture is formed substantially simultaneously with formation of the second fluid mixture.
